SEQUENCE LISTING (29 sequences)

SEQ ID NO: 1 - protein, 12 aa, Homo sapiens
THRILRRLFNLC

SEQ ID NO: 2 - protein, 12 aa, synthetic construct
  VARIANT 9: note = Wherein the X at position 9 may be G, A, V, L, K, or I.
THRILRRLXNLC

SEQ ID NO: 3 - protein, 783 aa, Homo sapiens
MRWQCGTRFRGLRPAVAPWTALLALGLPGWVLAVSATAAAVVPEQHASVAGQHPLDWLLTDRGPFHRAQEYADFMERYRQGFTTRYRIYREFARWKVNNLALERKDFFSLPLPLAPEFIRNIRLLGRRPNLQQVTENLIKKYGTHFLLSATLGGEESLTIFVDKQKLGRKTETTGGASIIGGSGNSTAVSLETLHQLAASYFIDRESTLRRLHHIQIATGAIKVTETRTGPLGCSNYDNLDSVSSVLVQSPENKVQLLGLQVLLPEYLRERFVAAALSYITCSSEGELVCKENDCWCKCSPTFPECNCPDADIQAMEDSLLQIQDSWATHNRQFEESEEFQALLKRLPDDRFLNSTAISQFWAMDTSLQHRYQQLGAGLKVLFKKTHRILRRLFNLCKRCHRQPRFRLPKERSLSYWWNRIQSLLYCGESTFPGTFLEQSHSCTCPYDQSSCQGPIPCALGEGPACAHCAPDNSTRCGSCNPGYVLAQGLCRPEVAESLENFLGLETDLQDLELKYLLQKQDSRIEVHSIFISNDMRLGSWFDPSWRKRMLLTLKSNKYKPGLVHVMLALSLQICLTKNSTLEPVMAIYVNPFGGSHSESWFMPVNEGSFPDWERTNVDAAAQCQNWTITLGNRWKTFFETVHVYLRSRIKSLDDSSNETIYYEPLEMTDPSKNLGYMKINTLQVFGYSLPFDPDAIRDLILQLDYPYTQGSQDSALLQLIELRDRVNQLSPPGKVRLDLFSCLLRHRLKLANNEVGRIQSSLRAFNSKLPNPVEYETGKLCS

SEQ ID NO: 4 - protein, 12 aa, Mus musculus
MHRIVRRLFNLC

SEQ ID NO: 5 - protein, 13 aa, Rattus rattus
IVHRIVRRLFNLC

SEQ ID NO: 6 - protein, 12 aa, Sus scrofa
  REGION 1..12: note = Sequence also obtained from Macaca mulatta and Canis lupus.
THRIVRRLFNLC

SEQ ID NO: 7-8 - skipped (2 entries)

SEQ ID NO: 9 - protein, 12 aa, synthetic construct
AHRILRRLFNLC

SEQ ID NO: 10 - protein, 12 aa, synthetic construct
TARILRRLFNLC

SEQ ID NO: 11 - protein, 12 aa, synthetic construct
THAILRRLFNLC

SEQ ID NO: 12 - protein, 12 aa, synthetic construct
THRALRRLFNLC

SEQ ID NO: 13 - protein, 12 aa, synthetic construct
THRIARRLFNLC

SEQ ID NO: 14 - protein, 12 aa, synthetic construct
THRILARLFNLC

SEQ ID NO: 15 - protein, 12 aa, synthetic construct
THRILRALFNLC

SEQ ID NO: 16 - protein, 12 aa, synthetic construct
THRILRRAFNLC

SEQ ID NO: 17 - protein, 12 aa, synthetic construct
THRILRRLANLC

SEQ ID NO: 18 - protein, 12 aa, synthetic construct
THRILRRLFALC

SEQ ID NO: 19 - protein, 12 aa, synthetic construct
THRILRRLFNAC

SEQ ID NO: 20 - protein, 12 aa, synthetic construct
THRILRRLFNLA

SEQ ID NO: 21 - DNA, 21 bp, synthetic construct
  source 1..21: mol_type = other DNA
catgcagaacccacgacagta

SEQ ID NO: 22 - DNA, 21 bp, synthetic construct
  source 1..21: mol_type = other DNA
cctcacgcagcttgttgtcta

SEQ ID NO: 23 - DNA, 24 bp, synthetic construct
  source 1..24: mol_type = other DNA
tctcctgaagaggaagagaaacgg

SEQ ID NO: 24 - DNA, 20 bp, synthetic construct
  source 1..20: mol_type = other DNA
tctgcaacgcagacttctcg

SEQ ID NO: 25 - protein, 12 aa, synthetic construct
CRFIRNLHLLRT

SEQ ID NO: 26 - protein, 11 aa, synthetic construct
THRILRRLFNL

SEQ ID NO: 27 - protein, 16 aa, synthetic construct
KKTHRILRRLFNLCKR

SEQ ID NO: 28 - protein, 16 aa, synthetic construct
KKMHRIVRRLFNLCKR

SEQ ID NO: 29 - protein, 16 aa, synthetic construct
KKTHRIVRRLFNLCKR

(19) United States
(12) Patent Application Publication
(10) Pub. No.: US 2026/0035409 A1
(43) Pub. Date: Feb. 5, 2026

(54) BRINP2-DERIVED PEPTIDE COMPOSITIONS FOR TREATING OBESITY AND WEIGHT MANAGEMENT

(71) Applicant: The Board of Trustees of the Leland Stanford Junior University, Stanford, CA (US)

(72) Inventors: Katrin Jennifer SVENSSON, Palo Alto, CA (US); Laetitia VOILQUIN, Redwood City, CA (US)

(73) Assignee: The Board of Trustees of the Leland Stanford Junior University, Stanford, CA (US)

(21) Appl. No.: 19/100,905

(22) PCT Filed: Jul. 5, 2023
(86) PCT No.: PCT/US2023/026933

§ 371 Date: Feb. 3, 2025

Related U.S. Application Data
(60) Provisional application No. 63/370,200, filed on Aug. 2, 2022.

(51) Int. Cl. C07K 7/08; A61K 38/00; A61K 45/06; A61P 3/04

(52) U.S. Cl. CPC C07K 7/08; A61K 45/06; A61P 3/04; A61K 38/00

(57) ABSTRACT

Compositions and methods are provided for preventing or treating obesity and/or overweight, and managing body weight. It is shown herein that a peptide that is proteolytically cleaved from the parent protein BRINP2 is effective in reducing food intake and obesity in a mammal. The peptide is referred to herein as BRNP2 expression in as BRINP2-related peptide (BRP).

### INTRODUCTION

Obesity is associated with a significant decrease in life expectancy, by 5-10 years, increased mortality such as diabetes, cardiovascular disease, and increased cardiovascular disease mortality independently of other risk factors. In humans, as little as 5-10% weight loss is sufficient to improve hyperglycemia, triglyceridemia, and other co-morbidities. Most of the genes involved in energy balance discovered so far act by controlling hunger, satiety, and food intake, pointing to a deeper genetic regulation and heritability than was previously appreciated. Therefore, it is not surprising that behavioral and lifestyle changes as the only weight loss intervention have had limited success in the general population. While lifestyle factors, exercise, and gastric bypass result in initial weight loss, weight regain is common owing to counterregulatory mechanisms in increased appetite (estimated to 100 kcal/day) and lower energy expenditure (by 25 kcal/day) per kg of body weight lost. Based on these observations, reducing body weight by pharmacological approaches combined with lifestyle factors is likely to represent the most efficacious method to improve the numerous complications associated with obesity.

Recently, modified GLP-1 peptide analogs such as liraglutide and semaglutide have had transformative efficacy in reducing body weight in humans. Peptide hormones represent a class of small (<100 amino acids), low abundant, and bioactive peptides involved in regulating physiological processes such as food intake and body weight regulation, making them attractive targets for modulation of energy metabolism.

Traditionally, novel bioactive peptide hormones have been identified by biochemical purification from endocrine organs, including insulin, glucagon, and oxyntomodulin from pancreatic or gut extracts, and neuropeptide Y (NPY) and gonadotropin-releasing hormone (GnRH) from brain extracts. More recently, extensive proteomics and peptidomics efforts have demonstrated detection and quantitation of peptide hormones and neuropeptides from complex biological tissues or blood, but there are still significant challenges in detection owing to their low abundance. Furthermore, because many peptide hormones are synthesized as part of larger precursors further processed into active fragments by posttranslational endoproteolytic cleavage, their dynamic regulation are undetected by RNA sequencing analyses or conventional proteomic analyses. Thus, we know very little about other endogenous peptides that control feeding behavior and obesity development, and the extent to which these uncharacterized peptides contribute to modulation of energy balance remains unclear.

Peptide hormones represent a class of small peptides that regulate a wide range of physiological functions. Systematic efforts to identify and characterize secreted bioactive polypeptides have traditionally been hampered by their low abundance, small size, and the difficulty predicting their functions. These peptide hormones are of great interest for clinical use and the development of therapies, including treatment of obesity and related disorders.

### SUMMARY OF THE INVENTION

Compositions and methods are provided for preventing or treating obesity and/or overweight, and for managing body weight. It is shown herein that a peptide that is proteolytically cleaved from the parent protein BRINP2 is effective in reducing food intake and obesity in a mammal. The peptide is referred to herein as BRINP2-related peptide (BRP). The encoded human peptide has the sequence THRILRRLFNLC (SEQ ID NO:1). BRP is endogenously secreted in human plasma and CSF and lowers food intake and body weight in vivo.

In some embodiments a composition is provided comprising or consisting of a BRP peptide of the formula (SEQ ID NO:2):

| | | | | | | | | | | | |
|---|---|---|---|---|---|---|---|---|---|---|---|
| T₁ | H₂ | R₃ | I₄ | L₅ | R₆ | R₇ | L₈ | X₉ | N₁₀ | L₁₁ | C₁₂ |

where X⁹ may be any amino acid. In some embodiments X⁹ is other than F, in some embodiments X⁹ is G, A, V, L, K, or I. When X⁹ is F, the peptide may comprise a non-naturally occurring modification.

In some embodiments, one or more residues in SEQ ID NO:2 are amidated, including without limitation, the C-terminal carboxyl group, the C₁₂ thiol, and any of R₃, R₆ and R₇. In some embodiments one or more residues are acylated, and may comprise a fatty acid. In some embodiments an acyl moiety is present at one or more of T₁, H₂, R₇, X₉ and L₁₁. In some embodiment the acyl moiety is a linear or branched C₄-C₂₀ alkyl, and may be a C₁₀-C₁₈ alkyl, particularly a C₁₆ alkyl, optionally substituted with halo, hydroxy, alkoxy, amino, alkylamino, dialkylamino, sulfate, or phosphate, and which may by saturated, or mono- or di-unsaturated. Fatty acids of interest for modification of the peptide include, without limitation, palmitic acid; stearic acid; arachidic acid; lauric acid; myristic acid; myristoleic acid; palmitoleic acid; sapienic acid; oleic acid; linoleic acid; α-linolenic acid; arachidonic acid; eicosapentaenoic acid; erucic acid; docosahexaenoic acid; etc., and in some embodiments is palmitic acid.

In some embodiments the peptide of SEQ ID NO:2 is modified by pegylation, glycosylation, conjugation to large proteins such as albumin, conjugation to immunoglobulin Fc, or conjugation with polymers. In some embodiments the peptide of SEQ ID NO:2 comprises amino acid modifications, such as the use of D-amino acid or beta amino acids, to increase the biological half-life. In some embodiments the peptide of SEQ ID NO:2 is truncated at the carboxy or amino terminus. In some embodiments a peptide of SEQ ID NO:2 is other than the naturally-occurring peptide.

The present disclosure provides for peptides of SEQ ID NO:2, pharmaceutical formulations comprising such peptides, and for methods of using such peptides. The peptides may comprise one or more of the modifications disclosed above. In particular, the present invention provides a pharmaceutical formulation comprising a therapeutically effective amount of a peptide of SEQ ID NO:2, alone or in combination with a pharmaceutically acceptable carrier. The formulation may be provided in a unit dose, comprising an effective dose of the peptide.

In some embodiments, a formulation is provided, comprising an effective dose of a BRP peptide of SEQ ID NO:2 and a pharmaceutically acceptable excipient, where the therapeutically effective amount of the BRP peptide is in the range of from about 0.1 mg/kg to about 100 mg/kg. In some embodiments the effective dose is at least about 0.1 mg/kg, at least about 0.5 mg/kg, at least about 1 mg/kg, at least about 5 mg/kg, at least about 10 mg/kg, at least about 20 mg/kg, at least about 50 mg/kg, up to about 100 mg/kg, in some embodiments the effective dose is from about 1 to 25 mg/kg. Dosing may be daily, every 2 days, every 3 or more days, e.g. weekly, semi-weekly, bi-weekly, monthly, etc. Dosing may be parenteral, including sustained release formulations. BRP and analogs thereof have the advantage of only reducing food intake and obesity without affecting insulin secretion. This drug may therefore be applicable to a large number of patients.

Further provided is a method for weight management, which may be associated with treating or delaying the progression or onset of diabetes and metabolic syndrome, especially type II diabetes, which include reducing complications of diabetes such as retinopathy, neuropathy, nephropathy and delayed wound healing, and related diseases such as insulin resistance (impaired glucose homeostasis), hyperglycemia, hyperinsulinemia, elevated blood levels of fatty acids or glycerol, hyperlipidemia including hypertriglyceridemia, Syndrome X, atherosclerosis and hypertension, wherein a therapeutically effective amount of a peptide of SEQ ID NO:2 is administered to a mammalian, e.g., human, patient in need of treatment.

In some embodiments, a method is provided for treating obesity and related diseases as defined herein, wherein a therapeutically effective amount of a combination of a peptide compound of SEQ ID NO:2 is administered to a mammalian, e.g., human, patient in need of treatment. In some such embodiments, the reduced food intake observed with administration of BRP is associated with weight loss, e.g. loss of 1% body weight, 2.5%, 5%, 7.5%, 10%, 12.5%, 15%, 17.5%, 20%, 22.5%, 25%, 27.5%, 30% or more, depending on the initial weight of the subject.

These methods of treatment with a peptide of SEQ ID NO:2 may be combined with one or more other types of therapeutic agent, such as an antidiabetic agent, a hypolipidemic agent or anti-obesity agent, e.g. metformin, sulfonylureas, e.g. glyburide, glipizide, glimepiride; glinides, e.g. repaglinide and nateglinide; thiazolidinediones, e.g. rosiglitazone, pioglitazone; DPP-4 inhibitors, e.g. sitagliptin, saxagliptin, linagliptin; GLP-1 receptor agonists, e.g. exenatide, liraglutide, semaglutide; SGLT2 inhibitors, e.g. canagliflozin, dapagliflozin, empagliflozin, etc. is administered to a human patient in need of treatment. The other therapeutic agents, when employed in combination with the compounds of the present invention may be used, for example, in those amounts indicated in the Physician's Desk Reference, as in the patents set out above or as otherwise determined by one of ordinary skill in the art.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1K. Sequence pattern recognition predicts small, secreted human polypeptide hormones expressed across human tissues. A. Prohormone processing. B. Bioinformatics flow diagram. C. Number of prohormones v. cleavage spacing. D. Number of prohormones v. number of cleavage sites. E. Cleavage site density of all human secreted proteins. F. Tissue distribution of prohormones. G-K. Tissue distribution of cleavage sites.

FIGS. 2A-2G. A 12-mer BRINP2 peptide, BRP, induces cfos expression and acutely suppresses food intake in mice. A. Peptide library design and bioactivity assays. B. NS-1 and INS1 cells treated for 1 h with vehicle, GLP-1 and NGF (positive controls), or 100 novel peptides at 100 μg/ml. N=3 biological replicates/group. Boxed area represents hits with >5-fold upregulation across both cell lines. C. Validation of the top hit, 100 μg/ml BRNP2_5 side-by-side with a scrambled BRNP2_5 peptide. D-E. Food intake in lean mice for up to 6 hours after I.P. injection of 5 mg/kg peptides, or 2 mg/kg GLP-1, N=3 mice/group. F. Food intake in DIO mice for up to 6 hours after I.P. injection of 10 mg/kg BRNP2_5 peptide, or 2 mg/kg GLP-1, N=3 mice/group. G. Fasting blood glucose levels in lean mice after I.P. injection of 5 mg/kg peptides or 2 mg/kg GLP-1, N=3 mice/group. Data are presented as S.E.M. *P<0.05, **P<0.01, ***P<0.001, ****P<0.0001 by one-way Anova (C) or two-way Anova for multiple comparisons (D-G).

FIGS. 3A-3H. BRP, but not the scrambled peptide, acutely suppresses food intake and meal size without affecting energy expenditure, locomotor activity, or anxiety-like behavior in mice. A-G. Analysis using metabolic cages for food intake (A-B), meal size (C), meal frequency (D), respiratory exchange ratio (RER) (E), VO2 (F), and ambulatory activity (G) in mice after a single injection of vehicle or 5 mg/kg BRP or 5 mg/kg BRP scrambled peptide. N=4 mice/group. H. Distance traveled and time spent in the center of an open-field assay in mice 30 minutes after a single injection of vehicle, or 5 mg/kg BRP.

FIGS. 4A-4F. BRP is endogenously circulating in human cerebrospinal fluid and plasma. A. Expression of BRNP2 prohormone across human organs (data from Human Protein Atlas). B. LC-MS (MS1 spectra) demonstrating the detection of endogenous BRP in human plasma at 27 minutes. Black=synthesized BRP standard. Red=endogenous BRP peptide. Bottom graph is zoomed in from top MS1 spectra. C. LC-MS (MS1 spectra) demonstrating the detection of endogenous BRP in human cerebrospinal fluid at 17 minutes. Black=synthesized BRP standard. Red=endogenous BRP peptide. Right graph is zoomed in from left MS1 spectra. D. Phylogenetic tree of BRNP2 parent protein across species. E. BRP sequences across species. F. NS-1 cells treated for 1 h with vehicle, NGF (positive control) or BRP peptide from different species (100 μg/ml).

FIGS. 5A-5C. Amino acid residues 3 and 8 are required for full BRP activity. A. cfos expression in NS1 cells in response to unmodified and amidated BRP. Neuronal growth factor (NGF) is used as a positive control. Data are presented as S.E.M. N=3 biological samples/group. B. Alanine substitutions in the BRP sequence followed by measurements of cfos activation in NS-1 cells after 1 h of treatment (100 μg/kg). Data are presented as S.E.M. N=3 biological samples/group. C. Alanine substitutions in the BRP sequence followed by food intake measurement in mice for up to 6 hours after I.P. injection of 5 mg/kg peptides, N=3 mice/group.

FIG. 6A-6L. BRP reverses obesity, diabetes, and hepatic steatosis in diet-induced obese mice. A-D. Accumulated food intake (a) and body weight change (b), total body weight (c-d) in mice during and after 14 days of treatment with vehicle, 100 μg/kg liraglutide, or 5 mg/kg BRP. N=10 mice/group. E-F. Glucose tolerance test (GTT) (e) and insulin tolerance test (ITT) (f) after 14 days of treatment with vehicle, 100 μg/kg liraglutide, or 5 mg/kg BRP. N=10 mice/group. G. Fasting insulin levels after 14 days of treatment with vehicle, 100 μg/kg liraglutide, or 5 mg/kg BRP. N=10 mice/group. H-K. White adipose tissue (h), brown adipose tissue (i), liver (j), skeletal muscle (k) weights after 14 days of treatment with vehicle, 100 μg/kg liraglutide, or 5 mg/kg BRP. N=10 mice/group. L. Histological analyses of white adipose tissue, brown adipose tissue, and liver after 14 days of treatment with vehicle, 100 μg/kg liraglutide, or 5 mg/kg BRP. Representative pictures of N=10 mice/group. Data are presented as S.E.M. *P<0.05, **P<0.01, ***P<0.001, ****P<0.0001 by two-way Anova for multiple comparisons (a-d).

FIG. 7A-7J. Sequence pattern recognition predicts small, secreted human polypeptide hormones expressed across human tissues. a-j. Detected prohormones and the number of cleavage sites per prohormone with no tissue annotation (a), wide tissue expression (b), in liver (c), blood (d), adipose tissue (e), parathyroid gland (f), salivary gland (g), kidney (h), thyroid gland (i) and skeletal muscle (j) Blue: known prohormones. Grey: unannotated functions.

FIG. 8A-8F. Characterization of synthetic peptides with LC-MS and LC-MS/MS. A-F. LC-MS (left) and LC-MS/MS (right) graphs for the 6 peptides tested in mice: BRNP2_5 (a), EDIL3_4 (b), FGF3_4 (c), FGF5_5 (d), FSTL4_3 (e) and SCG1_9 (f).

FIG. 9A-9F. GLP-1 acutely suppresses food intake and meal size without affecting energy expenditure or locomotor activity. a-f. Analysis using metabolic cages for food intake (a), meal size (b), meal frequency (c), respiratory exchange ratio (RER) (d), VO2 (e), and ambulatory activity (f) in mice after a single injection of vehicle or 2 mg/kg GLP-1. N=4 mice/group.

### DESCRIPTION OF THE SPECIFIC EMBODIMENTS

The invention provides novel uses and analogs of BRNP2-related peptide (BRP).

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Peptides

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

As used herein, the terms “peptide residue” and “peptidic structure” are intended to include peptides comprised of naturally-occurring L-amino acids and the corresponding D-amino acids, as well as peptide derivatives, peptide analogues and peptidomimetics of the naturally-occurring L-amino acid, structures. Approaches to designing peptide analogues, derivatives and mimetics are known in the art. For example, see Veber and Freidinger 1985 TINS p. 392; Evans, et al. 1987 J. Med. Chem. 30:229. Peptidomimetics that are structurally similar to therapeutically useful peptides may be used to produce an equivalent or enhanced therapeutic or prophylactic effect, by methods known in the art and further described in the following references: Spatola, A. F. 1983 in: Chemistry and Biochemistry of Amino Acids, Peptides, and Proteins, B. Weinstein, eds., Marcel Dekker, New York, p. 267; Holladay, et al. 1983 Tetrahedron Lett. 24:4401-4404.

Systematic substitution of one or more amino acids of a consensus sequence with a D-amino acid of the same type (for example, D-lysine in place of L-lysine) may be used to generate more stable peptides. In addition, constrained peptides comprising a consensus sequence or a substantially identical consensus sequence variation may be generated by methods known in the art (Rizo, et al. 1992 Ann. Rev. Biochem. 61:387, incorporated herein by reference in their entireties); for example, by adding internal cysteine residues capable of forming intramolecular disulfide bridges which cyclize the peptide, adding cyclic lactam bridge, or the use of flexible 6-aminohexanoic acid (Ahx), rigid aminoisobutyric acid (Aib) or D-amino acid residues to alter the stability of the helix.

As used herein, a “derivative” of a compound, for example, a peptide or amino acid, refers to a form of that compound in which one or more reactive groups in the compound have been derivatized with a substituent group. Examples of peptide derivatives include peptides in which an amino acid side chain, the peptide backbone, or the amino- or carboxy-terminus has been derivatized (for example, peptidic compounds with methylated amide linkages or hydroxylated amino acids or amino acid residues).

As used herein an “analog” of a compound refers to a compound which retains chemical structures of the reference compound necessary for functional activity of that compound yet which also contains certain chemical structures which differ from the reference compound. As used herein, a “mimetic” of a compound refers to a compound in which chemical structures of the referenced compound necessary for functional activity of that compound have been replaced with other chemical structures that mimic the conformation of the referenced compound. Examples of peptidomimetics include peptidic compounds in which the peptide backbone is substituted with one or more benzodiazepine molecules, peptides in which all L-amino acids are substituted with the corresponding D-amino acids and “retro-inverso” peptides (see U.S. Pat. No. 4,522,752 by Sisto, James, G. L. et al. 1993 Science 260:1937-1942, and Goodman et al. 1981 Perspectives in Peptide Chemistry pp. 283-294). Other derivatives include C-terminal hydroxymethyl derivatives, 0-modified derivatives (for example, C-terminal hydroxymethyl benzyl ether) and N-terminally modified derivatives including substituted amides such as alkylamides and hydrazides.

As used herein, the term “amino acid structure” is intended to include the amino acid, as well as analogues, derivatives and mimetics of the amino acid that maintain the functional activity of the compound. For example, the term “phenylalanine structure” is intended to include phenylalanine as well as pyridylalanine and homophenylalanine. The term “leucine structure” is intended to include leucine, as well as substitution with valine, isoleucine or other natural or non-natural amino acid having an aliphatic side chain, such as norleucine.

The amino- and/or carboxy-terminus of the peptide compounds disclosed herein can be standard amino and carboxy termini as seen in most proteins. Alternatively, the amino- and/or carboxy-terminus of the peptide compound can be chemically altered by the addition or replacement of a derivative group. Amino-derivative groups which can be present at the N-terminus of a peptide compound include acetyl, aryl, aralkyl, acyl, epoxysuccinyl and cholesteryl groups. Carboxy-derivative groups which can be present at the C-terminus of a peptide compound include alcohol, aldehyde, epoxysuccinate, acid halide, carbonyl, halomethane, diazomethane groups and carboxamide.

As used herein, “modified” refers to a polypeptide which retains the overall structure of a related polypeptide but which differs by at least one residue from that related polypeptide. As used herein a “modified C-terminus” is a C-terminus of a polypeptide that has a chemical structure other than a standard peptide carboxy group, an example of such a modified C-terminus being a C-terminal carboxamide.

As used herein, “pharmaceutically acceptable carrier” refers to a carrier medium which does not interfere with the effectiveness of the biological activity of the active ingredients and which is not toxic to the host or patient.

As used herein, the terms “peptide residue” and “peptidic structure” are intended to include peptides comprised of naturally-occurring L-amino acids and the corresponding D-amino acids, as well as peptide derivatives, peptide analogues and peptidomimetics of the naturally-occurring L-amino acid structures. Approaches to designing peptide analogues, derivatives and mimetics are known in the art (see Farmer, P. S. in: Drug Design E. J. Ariens, ed. Academic Press, New York, 1980, vol. 10, pp. 119-143; Ball J. B. & Alewood, P. F. 1990/. Mol. Recognition 3:55; Luthman, et al. 1996 A Textbook of Drug Design and Development, 14:386-406, 2nd Ed., Harwood Academic Publishers; Joachim Grante, Angew. 1994 Chem. Int. Ed. Engl. 33: 1699-1720). Peptidomimetics that are structurally similar to therapeutically useful peptides may be used to produce an equivalent or enhanced therapeutic or prophylactic effect, by methods known in the art (see Spatola, A. F. 1983 in: Chemistry and Biochemistry of Amino Acids, Peptides, and Proteins, B. Weinstein, eds., Marcel Dekker, New York, p. 267; Spatola, A. F. 1983 Vega Data, Vol. 1, Issue 3, Peptide Backbone Modifications (general review); Jennings-White, et al. 1982 Tetrahedron Lett. 23:2533; Holladay, et al. 1983 Tetrahedron Lett. 24:4401-4404; and Hruby, 1982 Life Sci. 31: 189-199).

In addition, constrained peptides may be generated by methods known in the art (Rizo, et al. 1992 Ann. Rev. Biochem. 61:387); for example, by adding internal cysteine residues or organic linkers capable of forming intramolecular bridges which cyclize the peptide, adding cyclic lactam bridge, or the use of flexible 6-aminohexanoic acid (Ahx), rigid aminoisobutyric acid (Aib) or D-amino acid residues to alter the stability of the helix.

Synthetic or non-naturally occurring amino acids refer to amino acids which do not naturally occur in vivo but which, nevertheless, can be incorporated into the peptide structures described herein.

BRINP2 (BMP/retinoic acid-inducible neural-specific protein 2) is one of a family of proteins predominantly and widely expressed in both the central nervous system (CNS) and peripheral nervous system (PNS). These proteins are believed to inhibit neuronal cell proliferation by negative regulation of the cell cycle G1/S transition, but do not have a well-characterized biological function. The refseq for human BRINP2 may be accessed at Genbank, NP_066988, and has the reference sequence (SEQ ID NO:3)

MRWQCGTRFR GLRPAVAPWT ALLALGLPGW VLAVSATAAA VVPEQHASVA GQHPLDWLLT
DRGPFHRAQE YADEMERYRQ GFTTRYRIYR EFARWKVNNL ALERKDFFSL PLPLAPEFIR
NIRLLGRRPN LQQVTENLIK KYGTHFLLSA TLGGEESLTI FVDKQKLGRK TETTGGASII
GGSGNSTAVS LETLHQLAAS YFIDRESTLR RLHHIQIATG AIKVTETRTG PLGCSNYDNL
DSVSSVLVQS PENKVQLLGL QVLLPEYLRE RFVAAALSYI TCSSEGELVC KENDCWCKCS
PTFPECNCPD ADIQAMEDSL LQIQDSWATH NRQFEESEEF QALLKRLPDD RFLNSTAISQ
FWAMDTSLQH RYQQLGAGLK VLFKKTHRIL RRLFNICKRC HRQPRFRLPK ERSLSYWWNR
IQSLLYCGES TFPGTFLEQS HSCTCPYDQS SCQGPIPCAL GEGPACAHCA PDNSTRCGSC
NPGYVLAQGL CRPEVAESLE NFLGLETDLQ DLELKYLLQK QDSRIEVHSI FISNDMRLGS
WFDPSWRKRM LLTLKSNKYK PGLVHVMLAL SLQICLTKNS TLEPVMAIYV NPFGGSHSES
WFMPVNEGSF PDWERTNVDA AAQCQNWTIT LGNRWKIFFE TVHVYLRSRI KSLDDSSNET
IYYEPLEMTD PSKNLGYMKI NTLQVFGYSL PFDPDAIRDL ILQLDYPYTQ GSQDSALLQL
IELRDRVNQL SPPGKVRLDL FSCLLRHRLK LANNEVGRIQ SSLRAFNSKL PNPVEYETGK
LCS

As demonstrated herein, a peptide that is proteolytically cleaved from BRINP2 is effective in reducing food intake and obesity in a mammal. The peptide is referred to herein as BRINP2-related peptide (BRP). The encoded human peptide has the sequence THRILRRLFNLC (SEQ ID NO:1). The BRP peptide is highly conserved across mammalian species, for example Homo sapiens, SEQ ID NO:1, THRILRRLFNLC; Mus musculus, SEQ ID NO:4, MHRIVRRLFNLC; Rattus rattus SEQ ID NO:5, IVHRIVRRLFNLC; Sus scrofa SEQ ID NO:6, THRIVRRLFNLC; Macaca mulatta, SEQ ID NO:7, THRIVRRLFNLC; Canis lupus, SEQ ID NO:8, THRIVRRLFNLC.

Applicants have further made targeted amino acid changes in the peptide, shown in, for example, SEQ ID NO:9, AHRILRRLFNLC; SEQ ID NO:10, TARILRRLFNLC; SEQ ID NO:11, THAILRRLFNLC; SEQ ID NO:12, THRALRRLFNLC; SEQ ID NO:13, THRIARRLFNLC; SEQ ID NO:14, THRILARLFNLC; SEQ ID NO:15, THRILRALFNLC; SEQ ID NO:16, THRILRRAFNLC; SEQ ID NO:17, THRILRRLANLC; SEQ ID NO:18, THRILRRLFALC; SEQ ID NO:19, THRILRRLFNAC; SEQ ID NO:20, THRILRRLFNLA. In some embodiments a peptide of the invention comprises or consists essentially of a peptide of SEQ ID NO:9-20.

In some embodiments a composition is provided, comprising a BRP peptide of the formula (SEQ ID NO:2):

| | | | | | | | | | | | |
|---|---|---|---|---|---|---|---|---|---|---|---|
| T₁ | H₂ | R₃ | I₄ | L₅ | R₆ | R₇ | L₈ | X₉ | N₁₀ | L₁₁ | C₁₂ |

where X⁹ may be any amino acid. In some embodiments X⁹ is other than F, in some embodiments X⁹ is G, A, V, L, K, or I, e.g. SEQ ID NO:17.

In some embodiments, one or more residues in SEQ ID NO:2 are amidated, including without limitation, the C-terminal carboxyl group, the C₁₂ thiol, and or any of R₃, R₆ and R₇. In some embodiments one or more residues are acylated, and may comprise a fatty acid. In some embodiments an acyl moiety is present at one or more of T₁, H₂, R₇, X₉ and L₁₁. In some embodiment the acyl moiety is a linear or branched C₄-C₂₀ alkyl, and may be a C₁₀-C₁₈ alkyl, particularly a C₁₆ alkyl, optionally substituted with halo, hydroxy, alkoxy, amino, alkylamino, dialkylamino, sulfate, or phosphate, and which may by saturated, or mono- or di-unsaturated. Fatty acids of interest include, without limitation, palmitic acid; stearic acid; arachidic acid; lauric acid; myristic acid; myristoleic acid; palmitoleic acid; sapienic acid; oleic acid; linoleic acid; α-linolenic acid; arachidonic acid; eicosapentaenoic acid; erucic acid; docosahexaenoic acid; etc, and may be palmitic acid.

In some embodiments the BRP peptide, optionally combined with amidation, and/or amino acid modifications, is modified by pegylation, glycosylation, conjugation to large proteins such as albumin, or conjugation with polymers.

In some embodiments the BRP peptide comprises amino acid modifications such as the use of D-amino acid or beta amino acids to increase the biological half-life.

In certain embodiments the BRP peptide is truncated at the carboxy or amino terminus. In some embodiments a peptide of SEQ ID NO:2 is other than the naturally-occurring peptide, and can vary in amino acid sequence or in modifications such as acylation, amidation, etc.

Optionally, peptides are modified by covalent linkage to a heterologous moiety, which may comprise a polymer, an Fc, an FcRn binding ligand, immunoglobulin, albumin, a collagen-binding motif, or by N-methylation. A covalently linked polymer may be selected from the group consisting of lipidation, as described above; a polyethyleneglycol (PEG) moiety; a polypropylenglycol (PPG) moiety; a PAS moiety; which is an amino acid sequence comprising mainly alanine and serine residues or comprising mainly alanine, serine, and proline residues, the amino acid sequence forming random coil conformation under physiological conditions [US No. 2010/0292130 and WO 2008/155134]; and a hydroxyethylstarch (HES) moiety [WO 02/080979]; an Fc immunonoglobulin sequence; a FcRn binding ligand; albumin and an albumin-binding ligand as well as an XTEN moiety (see Schellenberger, et al., 2009, Nature Biotechnology 27(12): 1186-1192).

An “Fc region” can be a naturally occurring or synthetic polypeptide that is homologous to an IgG C-terminal domain produced by digestion of IgG with papain. IgG Fc has a molecular weight of approximately 50 kDa. The BRP protein can be fused to the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases.

In other embodiments, BRP protein can comprise a polypeptide that functions as an antigenic tag, such as a FLAG sequence. FLAG sequences are recognized by biotinylated, highly specific, anti-FLAG antibodies, as described herein (see also Blanar et al., Science 256: 1014, 1992; LeClair et al., Proc. Natl. Acad. Sci. USA 89:8145, 1992). In some embodiments, the chimeric polypeptide further comprises a C-terminal c-myc epitope tag.

Where the covalent linkage is to PEG, the PEG molecular weight may be between about 1 kDa and about 100 kDa for ease in handling and manufacturing. For example, the PEG may have an average molecular weight of about 200, 500, 1000, 2000, 4000, 8000, 16,000, 32,000, 64,000, or 100,000 kDa. In some embodiments, the PEG may have a branched structure (U.S. Pat. No. 5,643,575; Morpurgo et al. Appl. Biochem. Biotechnol. 56:59-72 (1996); Vorobjev et al, Nucleosides Nucleotides 18:2745-2750 (1999); and Caliceti et al, Bioconjug. Chem. 10:638-646 (1999)).

Optionally, modified peptide derivatives comprise one or more substitutions of disulfide bonds with lactam bridges to increase the metabolic stability of the peptides. Cystathiones are resistant towards thiol reduction. Therefore, substitutions of disulfides with thioethers, or selenosulfide, diselenide and ditelluride bridges can provide protection against reduction [Knerr et al., ACS Chem Biol, 6(7), 753-760, 2011; Muttenthaler et al. J Med Chem., 53(24), 8585-8596, 2010]. Peptide disulfide bond mimics based on diaminodiacids can also be used to improve the stability of analogs (Cui et al., Angew Chem, 125, 9737-9741, 2013). The disulfide bridge can also be modified either by the insertion of linkers or bridges of a different nature.

Optionally, peptides are modified by the addition of one or more alkane, cholesterol, or PEG-cholesterol moieties to increase the metabolic stability of the peptides. Stapled peptides, via the introduction of a synthetic brace (staple), can be synthesized using ring-closing metathesis to lock a peptide in a specific conformation and reduce conformational entropy.

The sequence of the polypeptide may be altered in various ways known in the art to generate targeted changes in sequence. The polypeptide will usually be substantially similar to the sequences provided herein, i.e. will differ by at least one amino acid, and may differ by at least two but not more than about ten amino acids. The sequence changes may be substitutions, insertions or deletions, including truncation at the corboxy or the amino terminus. Scanning mutations that systematically introduce alanine, or other residues, may be used to determine key amino acids. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that do not alter primary sequence include chemical derivatization of polypeptides, e.g., acetylation, amidation, acylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. For examples, the backbone of the peptide may be cyclized to enhance stability (see Friedler et al. (2000) J. Biol. Chem. 275:23783-23789). Analogs of such polypeptides include those containing residues other than naturally occurring L-amino acids, e.g. D-amino acids or non-naturally occurring synthetic amino acids.

Those skilled in the art of peptide chemistry are aware that amino acid residues occur as both D and L isomers, and that the instant invention contemplates the use of either or a mixture of isomers for amino acid residues incorporated in the synthesis of the peptides described herein.

The present invention includes within its scope pharmaceutical compositions comprising, as an active ingredient, a therapeutically effective amount of at least one of the compounds of SEQ ID NO:2, alone or in combination with a pharmaceutical carrier or diluent. Optionally, compounds of the present invention can be used alone, in combination with other compounds of the invention, or in combination with one or more other therapeutic agent(s), e.g., an antidiabetic agent or other pharmaceutically active material.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The peptides described herein can be prepared by, for example, by using standard solid phase techniques. (See Merrifield, 1963. Am. Chem. Soc. 85:2149; J. M. Stewart and J. D. Young, 1984 Solid Phase Peptide Syntheses 2nd Ed., Pierce Chemical Company). These procedures can also be used to synthesize peptides in which amino acids other than the 20 naturally occurring, genetically encoded amino acids are substituted at one, two, or more positions of any of the modified peptides as disclosed herein. For instance, naphthylalanine can be substituted for tryptophan, facilitating synthesis. Other synthetic amino acids that can be substituted into the peptides of the present embodiments include L-hydroxypropyl, L-3, 4-dihydroxy-phenylalanyl, d amino acids such as L-d-hydroxylysyl and D-d-methylalanyl, L-a-methylalanyl, β-amino acids, and isoquinolyl. D amino acids and non-naturally occurring synthetic amino acids can also be incorporated into the peptides of the present embodiments (see Roberts, et al. 1983 Unusual Amino/Acids in Peptide Synthesis 5:341-449). In some embodiments, the naturally occurring side chains of the 20 genetically encoded amino acids, or any other side chain as disclosed herein can be transposed to the nitrogen of the amino acid, instead of the α-carbon as typically found in peptides.

The peptides can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively. Alternatively, in cases where a C-terminal amino alcohol is present, the C-terminal residue may be attached to 2-Methoxy-4-alkoxybenzyl alcohol resin (SASRIN™, Bachem Bioscience, Inc., King of Prussia, Pa.) as described herein and, after completion of the peptide sequence assembly, the resulting peptide alcohol is released with LiBH.sub.4 in THE (see J. M. Stewart and J. D. Young, supra, p. 92).

The syntheses of the peptides described herein can be carried out by using a peptide synthesizer, such as an Advanced Chemtech Multiple Peptide Synthesizer (MPS396) or an Applied Biosystems Inc. peptide synthesizer (ABI 433A). If the MPS396 was used, up to 96 peptides were simultaneously synthesized. If the ABI 433A synthesizer was used, individual peptides were synthesized sequentially. In both cases the stepwise solid phase peptide synthesis was carried out utilizing an Fmoc/t-butyl protection strategy.

Peptides with the desired purity can be obtained by purification using preparative HPLC, for example, on a Waters Model 4000 or a Shimadzu Model LC-8A liquid chromatograph. The solution of crude peptide is injected into a YMC S5 ODS column and eluted with a linear gradient of MeCN in water, both buffered with 0.1% TFA, using a flow rate of 14-20 mL/min with effluent monitoring by UV absorbance at 220 nm. The structures of the purified peptides can be confirmed by electro-spray MS analysis.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will be substantially pure, e.g. the peptide of interest will comprise at least 20% by weight of the desired product, more usually at least about 75% by weight, preferably at least about 95% by weight, and for therapeutic purposes, usually at least about 99.5% by weight, or more, in relation to contaminants related to the method of preparation of the product and its purification. The percentages may be based upon total protein.

### Methods of Treatment

Methods are provided for treating or delaying the progression or onset of diabetes and metabolic syndrome, especially type II diabetes, including complications of diabetes, including retinopathy, neuropathy, nephropathy and delayed wound healing, and related diseases such as insulin resistance (impaired glucose homeostasis), hyperglycemia, hyperinsulinemia, elevated blood levels of fatty acids or glycerol, obesity, hyperlipidemia including hypertriglyceridemia, Syndrome X, atherosclerosis and hypertension, and for increasing high density lipoprotein levels, wherein a therapeutically effective amount of a peptide of SEQ ID NO:2 is administered to a mammalian, e.g., human, patient in need of treatment, for a period of time sufficient to effect treatment.

Methods are provided for treating obesity and related diseases as defined herein, wherein a therapeutically effective amount of a peptide of SEQ ID NO:2 is administered to a mammalian, e.g., human, patient in need of treatment. In some such embodiments, the reduced food intake observed with administration of BRP is associated with weight loss, e.g. loss of 1% body weight, 2.5%, 5%, 7.5%, 10%, 12.5%, 15%, 17.5%, 20%, 22.5%, 25%, 27.5%, 30% or more, depending on the initial weight of the subject.

“In combination with”, “combination therapy” and “combination products” refers to the concurrent administration to a patient of the agents described herein. When administered in combination, each component can be administered at the same time or sequentially in any order at different points in time. Thus, each component can be administered separately but sufficiently closely in time so as to provide the desired therapeutic effect.

“Concomitant administration” of active agents in the methods of the invention means administration with the reagents at such time that the agents will have a therapeutic effect at the same time. Such concomitant administration may involve concurrent (i.e. at the same time), prior, or subsequent administration of the agents. A person of ordinary skill in the art would have no difficulty determining the appropriate timing, sequence and dosages of administration for particular drugs and compositions of the present invention.

“In combination with”, “combination therapy” and “combination products” refer, in certain embodiments, to the concurrent administration to a patient of the peptides described herein in combination with additional therapies. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

These methods of treatment are optionally combined with one or more other types of therapeutic agent, such as an antidiabetic agent, a hypolipidemic agent or anti-obesity agent, e.g. metformin, sulfonylureas, e.g. glyburide, glipizide, glimepiride; glinides, e.g. repaglinide and nateglinide; thiazolidinediones, e.g. rosiglitazone, pioglitazone; DPP-4 inhibitors, e.g. sitagliptin, saxagliptin, linagliptin; GLP-1 receptor agonists, e.g. exenatide, liraglutide, semaglutide; SGLT2 inhibitors, e.g. canagliflozin, dapagliflozin, empagliflozin, etc. is administered to a human patient in need of treatment. The other therapeutic agents, when employed in combination with the compounds of the present invention may be used, for example, in those amounts indicated in the Physician's Desk Reference, as in the patents set out above or as otherwise determined by one of ordinary skill in the art.

The term “obesity-related condition” refers to any disease or condition that is caused by or associated with (e.g., by biochemical or molecular association) obesity or that is caused by or associated with weight gain and/or related biological processes that precede clinical obesity. Examples of obesity-related conditions include, but are not limited to, type 2 diabetes, metabolic syndrome, fatty liver disease such as NASH, hyperglycemia, hyperinsulinemia, impaired glucose tolerance, impaired fasting glucose, hyperlipidemia, hypertriglyceridemia, insulin resistance, hypercholesterolemia, atherosclerosis, coronary artery disease, peripheral vascular disease, and hypertension.

Syndromes with associated obesity include, without limitation, 5p13 microduplication syndrome; 16p11.2 deletion; Albright hereditary osteodystrophy/PHP Type 1 a; Alstrom syndrome; Bardet Biedel syndrome (BBS); Borjeson-Forssman-Lehmann Syndrome; Carpenter syndrome; CHOPS syndrome; Chudley-Lowry syndrome; Cohen syndrome; Kabuki syndrome/Niikawa-Kuroki syndrome; Kleefstra syndrome; MORM syndrome; Prader-Willi Syndrome; Rubinstein-Taybi syndrome; Shashi-X-linked mental retardation; Smith Magenis Syndrome; WAGRO syndrome; OBHD; Ulnary Mammary syndrome; Bannayan-Riley-Ruvalcaba syndrome; Beckwith-Weidemann syndrome; Klippel-Trenaunay-Weber syndrome; Parkes Weber syndrome; Proteus syndrome; Silver-Russell syndrome; Simpson-Golabi-Behmel syndrome; Sotos syndrome; Weaver syndrome; Camera-Marugo-Cohen Syndrome; Clark-Baraitser Syndrome; MEHMO syndrome; MOMES syndrome; MOMO syndrome; Morgagni-Stewart-Morel Syndrome; 1p36 deletion syndrome; and 2p25.3 deletion syndrome. See, for example, Thaker (2017) Adolesc Med State Art Rev. 28(2): 379-405, herein specifically incorporated by reference.

Diabetes is a metabolic disease that occurs when the pancreas does not produce enough of the hormone insulin to regulate blood sugar (“type 1 diabetes mellitus”) or, alternatively, when the body cannot effectively use the insulin it produces (“type 2 diabetes mellitus”).

According to recent estimates by the World Health Organization, more than 200 million people worldwide have diabetes, whereby 90% suffer from type 2 diabetes mellitus. Typical long-term complications include development of neuropathy, retinopathy, nephropathy, generalized degenerative changes in large and small blood vessels and increased susceptibility to infection. Since individuals with type 2 diabetes still have a residual amount of insulin available in contrast to type 1 diabetic individuals, who completely lack the production of insulin, type 2 diabetes only surfaces gradually and is often diagnosed several years after onset, once complications have already arisen.

Insulin resistance occurs in 25% of non-diabetic, non-obese, apparently healthy individuals, and predisposes them to both diabetes and coronary artery disease. Hyperglycemia in type II diabetes is the result of both resistance to insulin in muscle and other key insulin target tissues, and decreased beta cell insulin secretion. Longitudinal studies of individuals with a strong family history of diabetes indicate that the insulin resistance precedes the secretory abnormalities. Prior to developing diabetes these individuals compensate for their insulin resistance by secreting extra insulin. Diabetes results when the compensatory hyperinsulinemia fails. The secretory deficiency of pancreatic beta cells then plays a major role in the severity of the diabetes.

Type II diabetes mellitus as diagnosed according to criteria published in the Report of the Expert Committee on the Diagnosis and Classification of Diabetes Mellitus whereby fasting plasma glucose level is greater than or equal to 126 milligrams per deciliter, and latent autoimmune diabetes mellitus of adults. It is characterized by insulin resistance and hyperglycemia, which in turn can cause retinopathy, nephropathy, neuropathy, or other morbidities. Additionally, diabetes is a well-known risk factor for atherosclerotic cardiovascular disease. Metabolic syndrome refers to a group of factors, including hypertension, obesity, hyperlipidemia, and insulin resistance (manifesting as frank diabetes or high fasting blood glucose or impaired glucose tolerance), that raises the risk of developing heart disease, diabetes, or other health problems; (Grundy et al, Circulation. 2004; 109:433-438).

There is a well-characterized progression from normal metabolic status to a state of impaired fasting glucose (IFG: fasting glucose levels greater than 100 mg/dL) or to a state of impaired glucose tolerance (IGT: two-hour glucose levels of 140 to 199 mg/dL after a 75 gram oral glucose challenge). Both IFG and IGT are considered pre-diabetic states, with over 50% of subjects with IFG progressing to frank type II diabetes within, on average, three years (Nichols, Diabetes Care 2007. (2): 228-233).

The term “metabolic syndrome” refers to metabolic disorders, particularly glucose and lipid regulatory disorders, including insulin resistance and defective secretion of insulin by pancreatic beta cells, and may further include conditions and states such as abdominal obesity, dyslipidemia, hypertension, glucose intolerance or a prothrombotic state, and which may further result in disorders such as hyperlipidemia, obesity, diabetes, insulin resistance, glucose intolerance, hyperglycemia, and hypertension.

The term “obesity” means the condition of excess body fat (adipose tissue), including by way of example in accordance with the National Institutes of Health Federal Obesity Clinical Guidelines for adults, whereby body mass index (“BMI”) calculated by dividing body mass in kilograms by height in meters squared is equal to or greater than twenty-five (25).

A compound, e.g. a peptide of SEQ ID NO:2, is effective to induce at least “minimal weight loss” when the compound induces, over a period of time from 12 to 52 weeks, a statistically significant and placebo-adjusted decrease in mean body weight of at least about 2.5%, but less than about 5.0%, in a cohort of subjects with a baseline mean BMI>27 kg/m².

A pharmaceutical composition is effective in “treatment of obesity” or “to induce weight loss” when the composition induces, over a period of time from 12 to 52 weeks, a statistically significant and placebo-adjusted decrease in body weight of at least about 5.0% in a cohort of subjects with a baseline mean BMI>27 kg/m².

It will be understood that there are medically accepted definitions of obesity and overweight. A patient may be identified by, for example, measuring body mass index (BMI), which is calculated by dividing weight in kilograms by height in meters squared, and comparing the result with the definitions. The recommended classifications for BMI in humans, adopted by the Expert Panel on the Identification, Evaluation and Treatment of Overweight and Obesity in Adults, and endorsed by leading organizations of health professionals, are as follows: underweight <18.5 kg/m², normal weight 18.5-24.9 kg/m², overweight 25-29.9 kg/m², obesity (class 1) 30-34.9 kg/m², obesity (class 2) 35-39.9 kg/m², extreme obesity (class 3)>40 kg/m² (Practical Guide to the Identification, Evaluation, and Treatment of Overweight and Obesity in Adults, The North American Association for the Study of Obesity (NAASO) and the National Heart, Lung and Blood Institute (NHLBI) 2000). Modifications of this classification may be used for specific ethnic groups.

Another alternative for assessing overweight and obesity is by measuring waist circumference. There are several proposed classifications and differences in the cutoffs based on ethnic group. For instance, according to the classification from the International Diabetes Federation, men having waist circumferences above 94 cm and women having waist circumferences above 80 cm are at higher risk of diabetes, dyslipidemia, hypertension and cardiovascular diseases because of excess abdominal fat. Another classification is based on the recommendation from the Adult Treatment Panel III where the recommended cut-offs are 102 cm for men and 88 cm for women. However, the methods, combinations and compositions of the invention may also be used for reduction of self-diagnosed overweight and for decreasing the risk of becoming obese due to life style, genetic considerations, heredity and/or other factors.

Dosage and frequency of dosing may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

An active agent can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants.

As noted above, an agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An agent can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds useful for co-administration with the active agents of the invention can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The active agents of the invention and/or the compounds administered therewith are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the active agents and/or other compounds can be achieved in various ways, usually by oral administration. The active agents and/or other compounds may be systemic after administration or may be localized by virtue of the formulation, or by the use of an implant that acts to retain the active dose at the site of implantation.

In pharmaceutical dosage forms, the active agents and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The term “sustained-release”, as in a sustained-release form, sustained-release composition or sustained-release formulation, is intended to include a form of an active ingredient, or formulation for an active ingredient, which has an extended in vivo half-life or duration of action. A sustained-release form may result from modification of the active ingredient, such as modifications that extend circulation residence time, decrease rates of degradation, decrease rates of clearance or the like, or may result from formulations or compositions which provide for extended release of the active ingredient, such as use of various liposomes, emulsions, micelles, matrices and the like. A controlled-release form or formulation is a type of sustained-release form or formulation.

In some embodiments a unit dose is at least about 0.1 mg/kg, at least about 0.5 mg/kg, at least about 1 mg/kg, at least about 5 mg/kg, at least about 10 mg/kg, at least about 20 mg/kg, at least about 50 mg/kg, at least about 100 mg/kg, in some embodiments the effective dose is from about 1 to 50 mg/kg. Dosing may be daily, every 2 days, every 3 or more days, e.g. weekly, semi-weekly, bi-weekly, monthly, etc. Dosing may be parenteral, including sustained release formulations. Dosing may be maintained for long periods of time, e.g. months, or years, to maintain desirable glucose and fatty acid levels.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of fatty liver disease in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Efficacy of treatment for obesity can be readily determined by weight low, for example the reduced food intake observed with administration of BRP is associated with weight loss, e.g. loss of 1% body weight, 2.5%, 5%, 7.5%, 10%, 12.5%, 15%, 17.5%, 20%, 22.5%, 25%, 27.5%, 30% or more, depending on the initial weight of the subject.

Along with weight loss, there can be an improvement in insulin sensitivity, although BRP by itself does not appear to directly alter insulin secretion. Insulin sensitivity can be monitored with various methods known to the art, to determine an improvement in insulin sensitivity (or decrease in insulin resistance), where an improvements may be, for example, 5%, 20%, 15%, 20%, 25%, 30%, 40%, 50% or more improvement. Hyperinsulinemic euglycemic clamp (HEC) is known to be the “gold standard” for the measurement of insulin sensitivity. However simplified assays can be used in quantification of insulin sensitivity. There are two major groups of insulin sensitivity indices: (1) Indices calculated by using fasting plasma concentrations of insulin, glucose and triglycerides, (2) indices calculated by using plasma concentrations of insulin and glucose obtained during 120 min of a standard (75 g glucose) OGTT. The former group include homeostasis model assessment-insulin resistance (HOMA-IR), QUIKI INDEX, and McAuley index while latter include, Matsuda, Belfiore, Cederholm, Avignon and Stumvoll index. For clinical uses HOMA-IR, QUIKI, and Matsuda are suitable while HES, McAuley, Belfiore, Cederholm, Avignon and Stumvoll index are suitable for epidemiological/research purposes.

The HEC-derived index of insulin sensitivity (ISI_{HEC}, ml/kg/min/μlU ml) is obtained during a steady state period of HEC. ISI_{HEC}=MCR/Iₘₑₐₙ where, Iₘₑₐₙ−average steady state plasma insulin response (μlU/ml), MCR: Metabolic clearance rate of glucose (ml/kg/min). MCR=Mₘₑₐₙ/(Gₘₑₐₙ×0.18), where Mₘₑₐₙ: Metabolized glucose expressed as average steady state glucose infusion rate per kg of body weight (mg/kg/min) Gₘₑₐₙ:Average steady state blood glucose concentration (mmol/I) 0.18-conversion factor to transform blood glucose concentration from mmol/I into mg/ml.

HOMA is a model of the relationship of glucose and insulin dynamics that predicts fasting steady-state glucose and insulin concentrations for a wide range of possible combinations of insulin resistance and β-cell function. The HOMA model has proved to be a robust clinical and epidemiological tool for the assessment of insulin resistance, where IR_{HOMA}=(I₀×G₀)!22.5(mathematically:e^{-Inx}=1/x).

Quantitative insulin sensitivity check index (QUICKI) is an empirically-derived mathematical transformation of fasting blood glucose and plasma insulin concentrations that provide a consistent and precise ISI with a better positive predictive power. It is a variation of HOMA equations, as it transforms the data by taking both the logarithm and the reciprocal of the glucose-insulin product, thus slightly skewing the distribution of fasting insulin values. It employs the use of fasting values of insulin and glucose as in HOMA calculations. QUICKI is virtually identical to the simple equation form of the HOMA model in all aspects, except that a log transform of the insulin glucose product is employed to calculate QUICKI. The QUICKI can be determined from fasting plasma glucose (mg/dl) and insulin (μlU/ml) concentrations.

McAuley index is used for predicting insulin resistance in normoglycemic individuals. Regression analysis was used to estimate the cut-off points and the importance of various data for insulin resistance (fasting concentrations of insulin, triglycerides, aspartate aminotransferase, basal metabolic rate (BMI), waist circumference). A bootstrap procedure was used to find an index most strongly correlating with insulin sensitivity index, corrected for fat-free mass obtained by HEC (Mffm/I).

Matsuda index derives an ISI from the OGTT. In these methods, the ratio of plasma glucose to insulin concentration during the OGTT is used. The OGTT ISI (composite) is calculated using both the data of the entire 3 h OGTT and the first 2 h of the test. The composite whole-body insulin sensitivity index (WBISI) is based on insulin values given in microunits per milliliter (μU/mL) and those of glucose, in milligrams per deciliter (mg/L) obtained from the OGTT and the corresponding fasting values.

Examples of suitable anti-diabetic agents for use in combination with the compounds of the present invention include biguanides (e.g., metformin or phenformin), glucosidase inhibitors (e.g, acarbose or miglitol), insulins (including insulin secretagogues or insulin sensitizers), meglitinides (e.g., repaglinide), sulfonylureas (e.g., glimepiride, glyburide, gliclazide, chlorpropamide and glipizide), biguanide/glyburide combinations (e.g., Glucovance®), thiazolidinediones (e.g., troglitazone, rosiglitazone and pioglitazone), PPAR-alpha agonists, PPAR-gamma agonists, PPAR alpha/gamma dual agonists, glycogen phosphorylase inhibitors, inhibitors of fatty acid binding protein (aP2), DPP-IV inhibitors, and SGLT2 inhibitors.

Thiazolidinediones include Mitsubishi's MCC-555 (disclosed in U.S. Pat. No. 5,594,016), Glaxo-Welcome's GL-262570, englitazone (CP-68722, Pfizer) or darglitazone (CP-86325, Pfizer, isaglitazone (MIT/J&J), JTT-501 (JPNT/P&U), L-895645 (Merck), R-119702 (Sankyo/WL), NN-2344 (Dr. Reddy/NN), or YM-440 (Yamanouchi).

Suitable PPAR alpha/gamma dual agonists include AR-H039242 (Astra/Zeneca), GW-409544 (Glaxo-Wellcome), KRP297 (Kyorin Merck) as well as those disclosed by Murakami et al, “A Novel Insulin Sensitizer Acts As a Coligand for Peroxisome Proliferation—Activated Receptor Alpha (PPAR alpha) and PPAR gamma. Effect on PPAR alpha Activation on Abnormal Lipid Metabolism in Liver of Zucker Fatty Rats”, Diabetes 47, 1841-1847 (1998), and in U.S. application Ser. No. 09/644,598, filed Sep. 18, 2000, employing dosages as set out therein, which compounds designated as preferred are preferred for use herein.

Suitable aP2 inhibitors include those disclosed in U.S. application Ser. No. 09/391,053, filed Sep. 7, 1999, and in U.S. application Ser. No. 09/519,079, filed Mar. 6, 2000, employing dosages as set out therein.

Suitable DPP4 inhibitors that may be used in combination with the compounds of the invention include those disclosed in WO99/38501, WO99/46272, WO99/67279 (PROBIODRUG), WO99/67278 (PROBIODRUG), WO99/61431 (PROBIODRUG), NVP-DPP728A (1-[[[2-[(5-cyanopyridin-2-yl)amino]ethyl]amino]acetyl]-2-cyano-(S)-pyrro-lidine) (Novartis) as disclosed by Hughes et al, Biochemistry, 38 (36), 11597-11603, 1999, TSL-225 (tryptophyl-1,2,3,4-tetrahydroisoquinoline-3-carboxylic acid (disclosed by Yamada et al, Bioorg. & Med. Chem. Lett. 8 (1998) 1537-1540, 2-cyanopyrrolidides and 4-cyanopyrrolidides, as disclosed by Ashworth et al, Bioorg. & Med. Chem. Lett., Vol. 6, No. 22, pp 1163-1166 and 2745-2748 (1996) employing dosages as set out in the above references.

Suitable meglitinides include nateglinide (Novartis) or KAD1229 (PF/Kissei).

Examples of other suitable glucagon-like peptide-1 (GLP-1,) compounds that may be used in combination with the GLP-1 mimics of the present invention include GLP-1(1-36) amide, GLP-1 (7-36) amide, GLP-1 (7-37) (as disclosed in U.S. Pat. No. 5,614,492 to Habener), as well as AC2993 (Amylin),LY-315902 (Lilly) and NN-2211 (NovoNordisk).

Examples of suitable hypolipidemic/lipid lowering agents for use in combination with the compounds of the present invention include one or more MTP inhibitors, HMG CoA reductase inhibitors, squalene synthetase inhibitors, fibric acid derivatives, ACAT inhibitors, lipoxygenase inhibitors, cholesterol absorption inhibitors, ileal Na.sup.+/bile acid cotransporter inhibitors, upregulators of LDL receptor activity, bile acid sequestrants, cholesterol ester transfer protein inhibitors (e.g., CP-529414 (Pfizer)) and/or nicotinic acid and derivatives thereof.

MTP inhibitors which may be employed as described above include those disclosed in U.S. Pat. Nos. 5,595,872, 5,739,135, 5,712,279, 5,760,246, 5,827,875, 5,885,983 and 5,962,440.

The HMG CoA reductase inhibitors which may be employed in combination with one or more compounds of SEQ ID NO:2 include mevastatin and related compounds, as disclosed in U.S. Pat. No. 3,983,140, lovastatin (mevinolin) and related compounds, as disclosed in U.S. Pat. No. 4,231,938, pravastatin and related compounds, such as disclosed in U.S. Pat. No. 4,346,227, simvastatin and related compounds, as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, cerivastatin, as disclosed in U.S. Pat. Nos. 5,006,530 and 5,177,080, atorvastatin, as disclosed in U.S. Pat. Nos. 4,681,893, 5,273,995, 5,385,929 and 5,686,104, atavastatin (Nissan/Sankyo's nisvastatin (NK-104)), as disclosed in U.S. Pat. No. 5,011,930, visastatin (Shionogi-Astra/Zeneca (ZD-4522)), as disclosed in U.S. Pat. No. 5,260,440, and related statin compounds disclosed in U.S. Pat. No. 5,753,675, pyrazole analogs of mevalonolactone derivatives, as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No.0142146 A2, and quinoline and pyridine derivatives, as disclosed in U.S. Pat. Nos. 5,506,219 and 5,691,322.

Hypolipidemic agents include pravastatin, lovastatin, simvastatin, atorvastatin, fluvastatin, cerivastatin, atavastatin and ZD-4522. In addition, phosphinic acid compounds useful in inhibiting HMG CoA reductase, such as those disclosed in GB 2205837, are suitable for use in combination with the compounds of the present invention.

Squalene synthetase inhibitors suitable for use herein include, but are not limited to, α-phosphono-sulfonates disclosed in U.S. Pat. No. 5,712,396, those disclosed by Biller et al, J. Med. Chem., 1988, Vol. 31, No. 10, pp 1869-1871, including isoprenoid (phosphinyl-methyl)phosphonates, as well as other known squalene synthetase inhibitors, for example, as disclosed in U.S. Pat. Nos. 4,871,721 and 4,924,024 and in Biller, S. A., Neuenschwander, K., Ponpipom, M. M., and Poulter, C. D., Current Pharmaceutical Design, 2, 1-40 (1996). Other squalene synthetase inhibitors suitable for use herein include the terpenoid pyrophosphates disclosed by P. Ortiz de Montellano et al, J. Med. Chem., 1977, 20, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes.

The fibric acid derivatives which may be employed in combination with one or more compounds of SEQ ID NO:2 include fenofibrate, gemfibrozil, clofibrate, bezafibrate, ciprofibrate, clinofibrate and the like, probucol, and related compounds, as disclosed in U.S. Pat. No. 3,674,836, probucol and gemfibrozil being preferred, bile acid sequestrants, such as cholestyramine, colestipol and DEAE-Sephadex (Secholex®, Policexide®), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives, such as disclosed in U.S. Pat. No. 4,759,923, quaternary amine poly(diallyldimethylammonium chloride) and ionenes, such as disclosed in U.S. Pat. No. 4,027,009, and other known serum cholesterol lowering agents.

The ACAT inhibitor which may be employed in combination with one or more compounds of SEQ ID NO:2 include those disclosed in Drugs of the Future 24, 9-15 (1999), (Avasimibe); “The ACAT inhibitor, CI-1011 is effective in the prevention and regression of aortic fatty streak area in hamsters”, Nicolosi et al, Atherosclerosis (Shannon, Irel). (1998), 137 (1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16 (1), 16-30; “RP 73163: a bioavailable alkylsulfinyl-diphenylimidazole ACAT inhibitor”, Smith, C., et al, Bioorg. Med. Chem. Lett. (1996), 6 (1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1 (3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8 (6), 359-62, or TS-962 (Taisho Pharmaceutical Co. Ltd).

The hypolipidemic agent may be an upregulator of LD2 receptor activity, such as MD-700 (Taisho Pharmaceutical Co. Ltd) and LY295427 (Eli Lilly). Examples of suitable cholesterol absorption inhibitor for use in combination with the compounds of the invention include SCH48461 (Schering-Plough), as well as those disclosed in Atherosclerosis 115, 45-63 (1995) and J. Med. Chem. 41, 973 (1998).

Examples of suitable ileal Na⁺/bile acid cotransporter inhibitors for use in combination with the compounds of the invention include compounds as disclosed in Drugs of the Future, 24, 425-430 (1999).

The lipoxygenase inhibitors which may be employed in combination with one or more compounds of SEQ ID NO:2 include 15-lipoxygenase (15-LO) inhibitors, such as benzimidazole derivatives, as disclosed in WO 97/12615, 15-LO inhibitors, as disclosed in WO 97/12613, isothiazolones, as disclosed in WO 96/38144, and 15-LO inhibitors, as disclosed by Sendobry et al “Attenuation of diet-induced atherosclerosis in rabbits with a highly selective 15-lipoxygenase inhibitor lacking significant antioxidant properties”, Brit. J. Pharmacology (1997) 120, 1199-1206, and Cornicelli et al, “15-Lipoxygenase and its Inhibition: A Novel Therapeutic Target for Vascular Disease”, Current Pharmaceutical Design, 1999, 5, 11-20.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable anti-obesity agents for use in combination with the compounds of the present invention include a NPY receptor antagonist, a MCH antagonist, a GHSR antagonist, a CRH antagonist, a beta 3 adrenergic agonist, a lipase inhibitor, a serotonin (and dopamine) reuptake inhibitor, a thyroid receptor beta drug and/or an anorectic agent.

The beta 3 adrenergic agonists which may be optionally employed in combination with compounds of the present invention include AJ9677 (Takeda/Dainippon), L750355 (Merck), or CP331648 (Pfizer,) or other known beta 3 agonists, as disclosed in U.S. Pat. Nos. 5,541,204, 5,770,615, 5,491,134, 5,776,983 and 5,488,064, with AJ9677, L750,355 and CP331648 being preferred.

Examples of lipase inhibitors which may be optionally employed in combination with compounds of the present invention include orlistat or ATL-962 (Alizyme), with orlistat being preferred.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Tissue-Based Human Prohormone Prediction Identifies an Anti-Obesity BRNP2-Derived Peptide

By using a computational reversed endocrine discovery approach, we predicted the existence of 2,683 novel secreted proteolytically cleaved peptides. Using this prediction, we generated and functionally tested a human hormone peptide library of small peptides to capture new biology, leading to the discovery of a secreted, highly evolutionary conserved 12-mer human brain peptide (BRP), derived from BRINP2, that circulates in human plasma and cerebrospinal fluid. BRP acts to acutely suppress food intake and meal size without affecting energy expenditure, locomotor activity, or anxiety-like behavior. Furthermore, chronic administration of BRP reverses obesity and diabetes in diet-induced obese mice, highlighting its potential as a new anti-obesity and anti-diabetic agent.

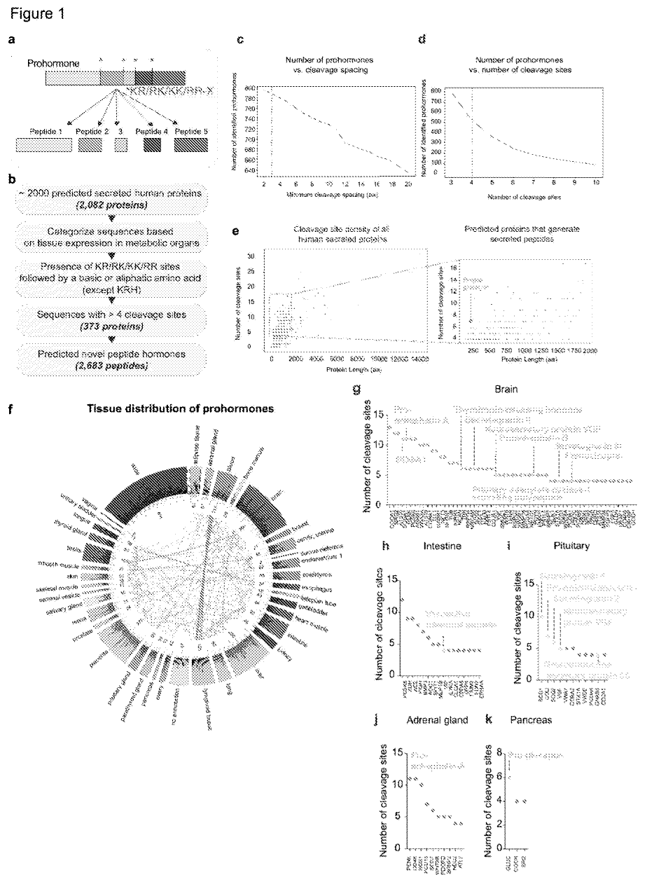

Sequence pattern recognition predicts small, secreted human peptide hormones and their expression across human tissues. The functional identification and characterization of small, proteolytically cleaved peptides have traditionally been challenging because of their low abundance and the ability to distinguish bioactive fragments from inactive fragments or degradation products. We therefore made use of the fact that peptide hormones are often synthesized as part of larger prohormones that are processed into peptides by posttranslational endoproteolytic cleavage, similar to GLP-1. This process occurs at specific dibasic amino acid residues, KR/RR/RK/KK, followed by a non-basic, non-aliphatic amino acid (KRH being an exception) (FIG. 1a). The proteolytic cleavage is mediated by enzymes present in the secretory pathway, including the subtilisin-like proprotein convertases furin, prohormone convertase 2 (PC2), and prohormone convertase 1/3 (PC1/PC3). Using these conserved sites as a criterion, we generated a code for sequence pattern recognition using Regular Expression (RegEx) for matching text patterns of protein sequences. Using this method, we are able to annotate all amino acid sequences with a signal peptide (2,082 proteins) to their tissue of origin (26), that also have more than 4 of any combination of the KR/RR/RK/KK cleavage sites (FIG. 1b). The space between cleavage sites was set to 3 to generate peptides >4 aa in length to exclude tripeptides (FIG. 1c). The minimum number of cleavage sites per protein was set to 4, based on the number of cleavage sites to generate at least five peptides after cleavage (FIG. 1d). In addition, the next criterion was that the protein needs to be smaller than 2,000 amino acids to exclude extremely long peptide sequences, to enrich for prohormones with high cleavage site density, such as pre-proglucagon (FIG. 1e). In total, out of 2,082 secreted proteins, we identify 373 prohormones predicted to generate 2,683 novel peptides, the majority of which are entirely unknown (FIG. 1b).

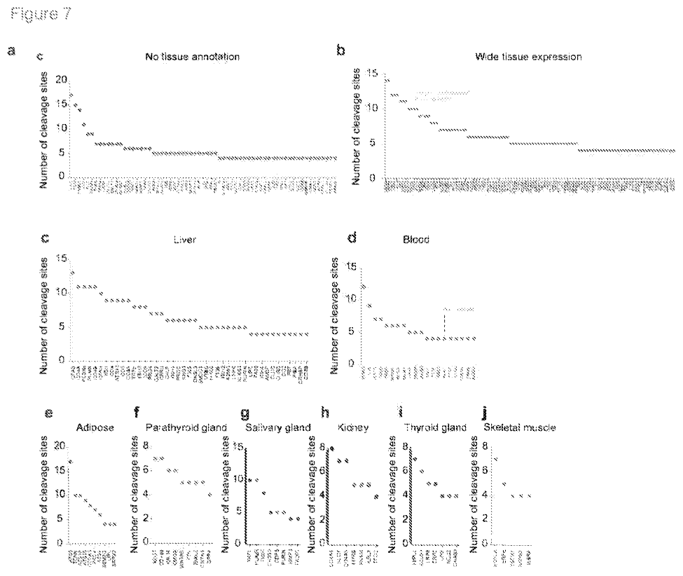

To analyze the tissue distribution of the identified prohormones, we categorized the prohormones according to distinct or shared tissue expression (FIG. 1f and FIG. 7). The largest group of peptides identified, 601 peptides, have wide expression (FIG. 7b), while the second largest number of peptides, 366, belong to the brain (FIG. 1f). Interestingly, the brain prohormone prediction accurately predicts 9 already known prohormones and their cleaved neuropeptides, and an additional 50 brain-enriched proteins with unknown functions (FIG. 1g). For example, we identify peptides derived from proenkephalin-A, pituitary adenylate cyclase activating polypeptide (PACAP), secretogranin-II, and thyrotropin-releasing hormone (TRH) (FIG. 1g). Furthermore, we identify many known peptides, including vasoactive intestinal peptide in the intestine (FIG. 1h), neuropeptide precursors in the pituitary gland, including secretogranin-1, pro-opiomelanocortin (POMC) (FIG. 1i) and proenkephalin-A in the adrenal gland (FIG. 1j). Lastly, we find that the pancreas is enriched for proglucagon, which validates that the computational approach can predict true peptide hormones (FIG. 1k). In conclusion, the brain (FIG. 1g) and liver (FIG. 7c) are major contributors to the release of small, secreted peptides, most of which have no previously annotated function.

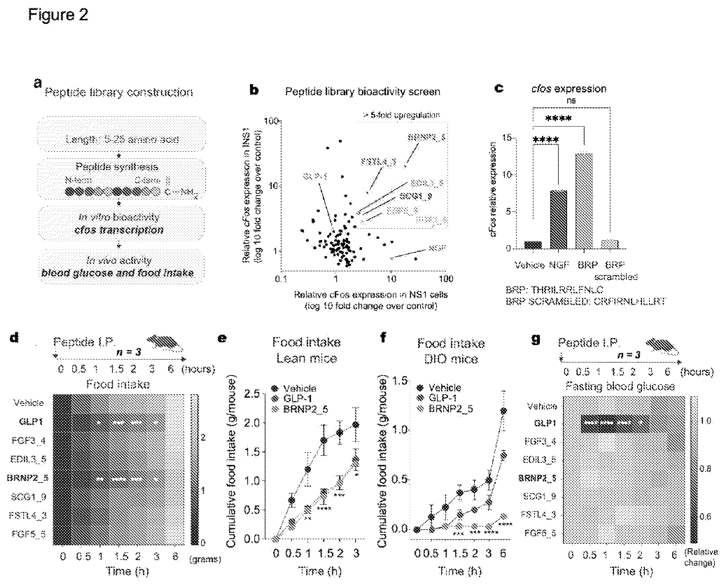

A 12-mer BRINP2 peptide, BRP induces cfos expression and acutely suppresses food intake in mice. To functionally test the bioactivity of a fraction of the identified peptides, we generated a peptide library of sequences with a size ranging from 5 to 25 amino acids in length (FIG. 2a). Proteins that are annotated as enzymes, non-extracellular matrix proteins, or have a transmembrane domain were excluded from this library. The novel peptides were analyzed with Protein BLAST (NCBI) and had no homology with any other protein than the original parent protein. The custom peptide library was generated by chemical peptide synthesis (see Methods).

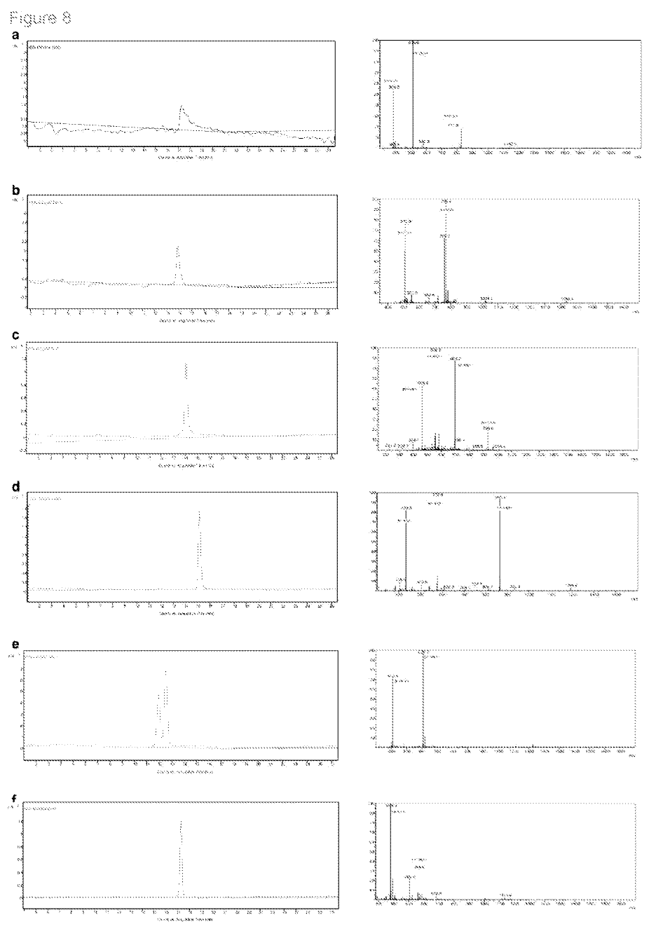

A feature of many known biologically active peptide hormones is the presence of an amidated C-terminus, which can be important for peptide bioactivity, as for gastrin-releasing peptide neuromedin-B, or for increased peptide stability, as for GLP-1. Therefore, the peptide library was constructed with an amide (NH2) group at the C-terminus as the only modification for comparative studies between peptides (FIG. 2a). In total, we generated a library of 100 peptides for functional biological screening. The peptide solubility was deduced according to its overall charge and reconstituted accordingly to optimal solubility. For all peptides, quality control by mass spectrometry was performed which validated the purity of the synthesized peptides for the primary screen according to their theoretical molecular weight (FIG. 8). Next, we functionally explored the potency of 100 of the peptides in activating cfos expression, a marker of cellular activity, after 1 hour of peptide treatment across two cell lines, Neuroscreen-1, NS-1 (rat neuronal origin) and INS1 cells (rat pancreatic origin) (FIG. 2b). Glucagon-like peptide 1 (GLP-1⁷⁻³⁷), a 30-mer peptide ligand for the glucagon receptor family of G protein-coupled receptors in pancreatic 3-cells, was used as a positive control at 30 μM in INS1 cells. NGF (Nerve Growth Factor) was used as a positive control at 2 nM in NS-1 cells. Expectedly, we find that GLP-1⁷⁻³⁷ induces a ˜4-fold elevation of cfos expression in INS1 cells and NGF induces a ˜10-fold induction of cfos expression in NS-1 cells (FIG. 2b). Across the 100 tested peptides, 6 peptides, FGF34, EDIL3_4, BRNP2_5, SCG1_9, FSTL4_3, and FGF5_5 induced more than a >5-fold cfos expression across both cell lines compared with vehicle.

Interestingly, a novel BRNP2-derived 12-mer secreted peptide derived from the parent protein BRNP2, induced cfos expression >10-fold across both cell lines (FIG. 2b). Importantly, a scrambled sequence of BRNP2_5 used at the same concentration and time points has no effect (FIG. 2c). The scrambled peptide was analyzed with Protein BLAST (NCBI) and did not match any known protein. These data demonstrate that the function of the BRNP2_5 peptide is specific and solely determined by the exact sequence of amino acids and excludes the possibility of other contaminants in the peptide solution.

We next performed in vivo studies to determine whether any of these peptides affected food intake or blood glucose levels—two biological consequences of cfos activation. For acute food intake studies, lean, 8-week-old C57BL/6 mice (N=3/group) were fasted for 16 hours prior to administration of vehicle (saline), GLP-1 at 2 mg/kg, or FGF3_4, EDIL3_4, BRNP2_5, SCG1_9, FSTL4_3, and FGF5_5 peptides at 5 mg/kg. The food intake was then monitored over 6 hours. Remarkably, we find that BRNP2_5 potently controls acute food intake starting at 30 minutes and lasting up to 3 hours in lean mice (FIG. 2d-e). Notably, the appetite-suppressing effects are comparable to GLP-1 and the largest difference in food intake was seen after 1.5 hours after injection (FIG. 2d-e). In diet-induced obese (DIO) mice, we find that BRNP2_5 have even stronger suppression of food intake relative vehicle and GLP-1 treated mice (FIG. 2f). Next, we performed a similar experiment in lean, 8-week-old C57BL/6 mice (N=3/group) but in the absence of food in order to measure whether any of the peptides acutely lowered blood glucose as an indication of increased insulin secretion. Upon peptide administration, only GLP-1 lowered blood glucose, while no significant difference was seen with any of the other peptides (FIG. 2g). These results suggest that the BRNP2_5 peptide reduces food intake without acutely lowering blood glucose through insulin-dependent or insulin-independent mechanisms. Based on these results, we named this peptide BRP, for BRNP2-Related Peptide.

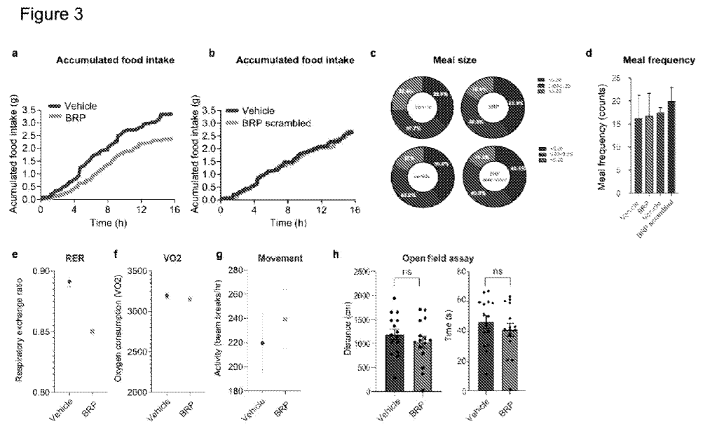

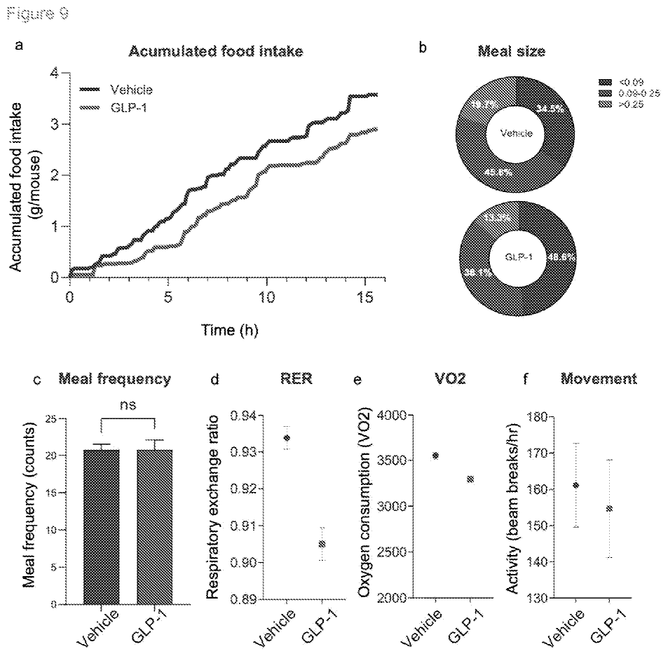

BRP acutely suppresses food intake and meal size without affecting energy expenditure, locomotor activity, or anxiety-like behavior. To study the metabolic effects in more detail, we next used metabolic cages performed with a Columbus comprehensive lab animal monitoring system. C57BL6/J mice were acclimated to the cages and randomized into two groups, followed by an I.P. injection with either vehicle (saline) or 5 mg/kg BRP (N=4/group). Again, BRP dramatically reduces food intake (FIG. 3a), an effect not seen when the scrambled BRP peptide is administered (FIG. 3b). Interestingly, we find that BRP, but not the scrambled BRP peptide, lowers meal size (FIG. 3c) without affecting meal frequency (FIG. 3d). BRP also acutely lowers respiratory exchange ratio (FIG. 3e), consistent with the increased oxidation of fats rather than carbohydrates when consuming smaller meals. On the other hand, acute BRP treatment does not alter oxygen consumption (VO₂) (FIG. 3f) or ambulatory activity (FIG. 3g). In addition, by performing an open-field assay, we show that BRP does not affect anxiety-like behavior in mice treated with BRP (FIG. 3h). In aggregate, these results establish BRP as a potent food intake suppressant (FIG. 3 and FIG. 9) and strongly support that BRP lowers appetite not by inducing anxiety, fatigue, or toxicity, but by reducing food intake.

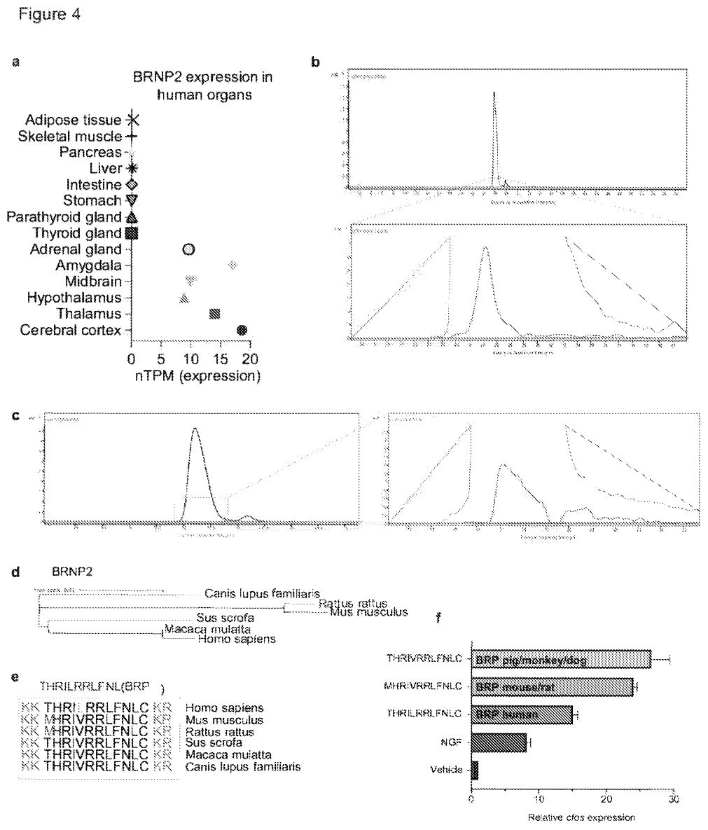

The unmodified BRP peptide is circulating in human cerebrospinal fluid and plasma. BRNP2 parent protein is highly expressed in the human brain (FIG. 4a) and was reported to be involved in neurodevelopmental disorders when embryonically deleted. Since the cleaved BRP peptide was predicted using a computational method, we next sought to establish whether BRP is endogenously secreted and detected in humans. By using targeted liquid chromatography-tandem mass spectrometry (LS-MS) analysis using the synthesized BRP as an internal standard, we quantitated the m/z intensity of the endogenous peptide relative to the spiked peptide of known concentration. Remarkably, we find that the intact BRP peptide is present in human plasma (FIG. 4b) and human cerebrospinal fluid (FIG. 4c). These data demonstrate that BRP is endogenously secreted which substantiated its biological relevance in humans. To further investigate BRP's translational potential, we performed sequence analysis and phylogenetic analyses of the parent BRNP2 protein, which shows that BRNP2 is highly conserved and secreted in all species (FIG. 4d-e). Furthermore, human, monkey and pig BRNP2 are more similar to each other than the rat, mouse, and dog sequence (FIG. 4d), and the cleavage site to generate the small secreted BRP peptide is conserved across all six species (FIG. 4e). Additionally, human and pig BRP only differs by one amino acid residue, Leu (L) at position 5, and demonstrated similar activity as human BRP (FIG. 4f). These results show that human BRP is a true bona fide secreted hormone with potential for therapeutic translation.

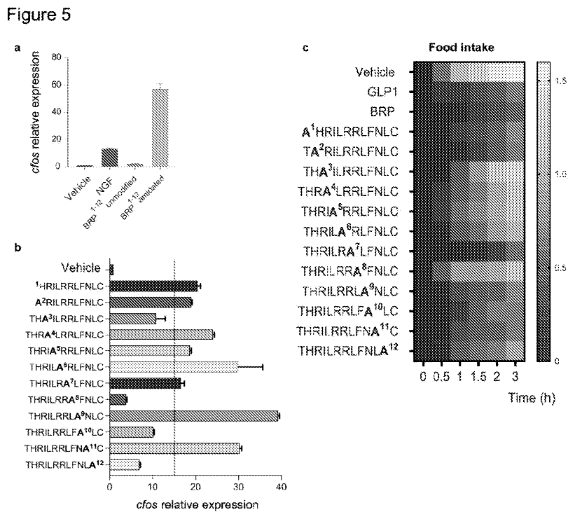

Arg³ and Leu⁸ are required for full BRPbioactivity. The endogenous BRP is a 12-mer peptide with the sequence SEQ ID NO:1 THRILRRLFNLC. A common feature of a large fraction of the known active peptide hormones is the presence of an amidated (NH2) C-terminus. This C-terminal amidation has been shown to be particularly important for peptide bioactivity, as for gastrin-releasing peptide neuromedin-B, or for increased peptide stability, as for GLP-1. Our preliminary data indeed confirms that the C-terminal amidation of BRP is critical for bioactivity, as the non-amidated BRP has no activity in cells (FIG. 5a). Furthermore, by synthesizing peptides with Ala substitutions at positions 1-12 to determine which amino acid residues are required for the bioactivity, we identify that amino acid residues 3, 8, 10 and 12 were important to retain full activity, while the other Ala substitutions did not impair activity (FIG. 5b). In these experiments, we used the minimal activity of >15-fold over vehicle as a cut-off to be considered fully active. Interestingly, we find that Ala substitution on residue 7 improves the appetite-suppressing effect of BRP while Ala substitution on residues 3, 4, 5, 6, 8 and 12 demonstrate a reduced efficacy in lowering food intake in mice (FIG. 5c).

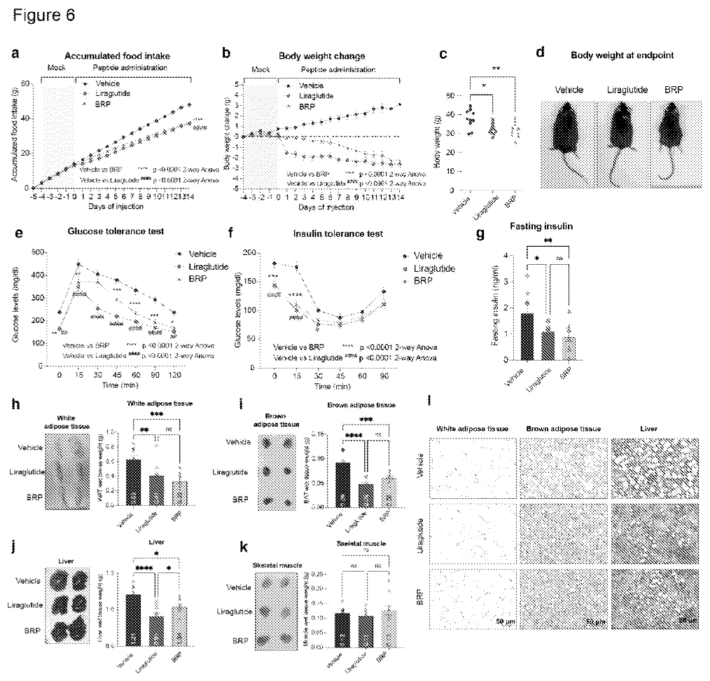

Chronic administration of the amidated BRP reverses obesity and diabetes in diet-induced obese mice. Given that a reduction in food intake by 20-50% is expected to dramatically reduce body weight, we next performed a chronic administration experiment in mice that had developed obesity and diabetes. 8-week-old C57BL6/J mice on 6 weeks of HFD were randomized into three groups followed by mock injections I.P. for 4 days, followed by treatments with either vehicle (saline), 100 μg/kg liraglutide, or 5 mg/kg BRP (N=10/group). After 14 days of daily injections, mice treated with BRP and liraglutide had significantly lower accumulated food intake compared with vehicle controls and demonstrated no desensitization over the course of the experiment (FIG. 6a). Furthermore, both BRP and liraglutide treatment groups resulted in significant and visible weight differences of an average of 4 grams with BRP and liraglutide compared with vehicle (FIG. 6b-d). Glucose and insulin tolerance tests at the endpoint showed that BRP mice had improved glucose tolerance (FIG. 6e) and insulin tolerance (FIG. 6f), an effect accompanied by both lower fasting glucose (FIG. 6e-f) and fasting insulin levels (FIG. 6g). In conclusion, chronic BRAP treatment reduces obesity and improves glucose homeostasis in mice. We next assessed whether the weight loss was associated with a loss of fat mass and/or lean mass. After 14 days, the BRP treatment group had significantly lower subcutaneous (inguinal) white adipose tissue mass (FIG. 6h), brown adipose tissue mass (FIG. 6i) and liver mass (FIG. 6j), comparable to the effect of liraglutide. There was no difference in skeletal muscle mass with either treatment (FIG. 6k). These data were consistent with histological analyses of the adipose and liver tissues that demonstrated that BRP reduced adipocyte size and ectopic lipid accumulation in the liver (FIG. 6l). In conclusion, our robust and reproducible preliminary data demonstrate that the novel, previously never identified BRP peptide has dramatic effects on lowering food intake, reversing obesity, and reversing diabetes in mice without any apparent adverse behavioral effects.

Metabolic diseases such as obesity have become a major public health concern. In 2020, the prevalence of obesity was >40% in the US. Obesity significantly increases the risk of type 2 diabetes, fatty liver disease, cardiovascular and pulmonary diseases, and musculoskeletal disorders. Lifestyle interventions are known to provide moderate efficacy because of complex and persistent hormonal, metabolic and neurochemical adaptations while medications to treat obesity are commonly associated with several side effects. The challenge is to find a drug that sustainably corrects excess weight while reducing comorbidities and adverse effects. Peptides have gained such considerable attention in the last decade that they are now part of the main strategies for developing new medicines. The peptide drug discovery field has revolutionized medicine with the introduction of over 60 peptide drugs approved in the US. To date, a number of peptides have been identified as modulators of food intake and obesity, including leptin, ghrelin, and glucagon-derived peptides. While peptide hormones traditionally have been identified by biochemical purification, our computational analysis has unlocked exciting possibilities to target selective aspects of metabolism via distinct mechanisms from current drugs.

Here, we find that a predicted peptide, BRP, is endogenously secreted in human plasma and CSF and lowers food intake and body weight in vivo. As BRP is a circulating ligand, identification of its receptor is of great interest to understand its mechanism of action and its intracellular signaling in the brain. Hormonal signaling through protein phosphorylation is one of the most important post-translational modifications allowing for rapid changes in cellular metabolic states, including feeding control. Many vital physiological processes are modulated by G-protein-coupled receptors (GPCRs) including GLP-1 but, whether BRP binds to a GPCR remains unknown. The fact that BRP induces cfos expression in vitro strongly suggest that BRP binds to a cell surface receptor to induce intracellular signaling cascades linked to food intake suppression. Given BRP's beneficial effects on food intake and body weight, our studies emphasize the unique opportunity for peptide engineering of BRP for therapeutic purposes which may offer certain advantages over current therapies.

### Materials and Methods

Preparation of samples for LC-MS analysis. Pooled human plasma (IPLALIH10 ML) and human Cerebrospinal fluid (CSF, IRHUCSF5 ML) were purchased from Innovative Research. 8 aliquots of 250 μL of plasma or CSF (2 mL total per preparation) was mixed with 750μL of 100 mM Tris-HCl (pH 8.2) and boiled at 95° C. for 10 minutes. 1 mM DTT was added, samples were vortexed and incubated for 50 minutes at 60° C. Iodoacetamide was added for a final concentration of 5 mM and incubated at RT for 1 hour in the dark. Formic acid was added to 0.2% final concentration. Samples were centrifuged at 15,000 rpm for 20 min. Supernatants were desalted and concentrated with C8 columns (Waters, WAT054965) and eluted with 100 μL of 80% ACN. Samples were centrifuged at 15,000 rpm for 10 min. Supernatant was transferred to a LC-MS vial.

Targeted measurements of BRP by LC-MS. Targeted metabolomics measurements were performed using an Agilent Q-TOF LC-MS instrument. MS analysis was performed using electrospray ionization (ESI) in positive mode. The dual ESI source parameters were set as follows: the gas temperature was set at 325° C. with a drying gas flow of 13 I min−1 and the nebulizer pressure at 30 psi; the capillary voltage was set to 4000 V; and the fragmentor voltage set to 185 V. The +3 ion of 533.64 was fragmented at 25 CE for the MSMS spectra. Mobile phases were as follows: buffer A 100% H2O+0.1% Formic Acid, and buffer B 90% ACN/10% H2O+0.1% Formic Acid. A 60-minute LC gradient from 95% A/5% B to 60% A/40% B was used.

Bioinformatics. To generate the program for sequence pattern recognition using Regular Expression (RegEx) for matching text patterns of protein sequences, we used the FASTA files for all reviewed, secreted, human genes (as in secreted.fasta) retrieved from UniProtKB API. The code for the Prohormone Predictor can be found at https://github.com/Svensson-Lab/pro-hormone-predictor. This program predicts whether a secreted gene has prohormone activity based on the number of cleavage sites it contains. For tissue distribution, prohormones were categorized according to distinct or shared tissue expression based on tissue expression data from Human Protein Atlas. The following criteria were used to annotate all predicted prohormones and their subsequent peptides: >4 of any combination of the KR/RR/RK/KK cleavage sites, >3 cleavage sites per protein, >4 cleavage sites per protein, prohormone <2,000 amino acids in size.

Mouse studies. Animal experiments were performed per procedures approved by the Institutional Animal Care and Use Committee of the Stanford Animal Care and Use Committee (APLAC) protocol number #32982. C57BL/6J male mice were purchased from the Jackson Laboratory (#000664) and were used after 1 week of acclimatization after import into the facility. Unless otherwise stated, all mice were in good health and housed in a temperature-controlled (20-22° C.) room on a 12-hour light/dark cycle with ad lib access to food and water.

For acute experiments, after overnight fasting for 16 hours, 8-week-old C57BL/6 mice were weighed and I.P. injected with either saline, GLP-1 at 2 mg/kg or peptides at 5 mg/kg (N=3 mice per group). Fasted blood glucose and food intake was then monitored for 6 hours.

Pharmacologic studies using BRP peptide were performed in mice with established diet-induced obesity. Male C57BL/6J mice purchased from Jax were fed a high-fat diet (#D12492, Research Diets) for 6 weeks. Mice were mock injected with saline for four days prior to peptide injections to prevent stress-induced weight loss. Mice were daily I.P. injected for 14 days with vehicle (saline) or indicated doses of liraglutide or BRP diluted in saline. Food intake and body weight were monitored every day. At the end of the experiments, mice and tissue weights were recorded. Tissues and plasma were collected and frozen for further analyses.

Cell culture. NS-1 rat neuronal cell line was cultured with RPMI 1640 medium (Gibco) supplemented with 10% FBS and 1% L-glutamine. INS-1 832/13 rat insulinoma cell line (#SCC207) was cultured with RPMI 1640 medium (Gibco) supplemented with 10% FBS, 1% L-glutamine, 10 mM HEPES, 1 mM NaPyruvate and 50 μM β-mercaptoethanol. For peptide activity assay, 3×10⁵ NS-1 and INS1 cells were plated in 12 well-plates. The next day, cells were washed twice with warm PBS and starved in serum-free RPMI overnight. Indicated concentrations of NGF (2 nM) or GLP-1 (30 μM) or peptides (100 μg/ml) were added and incubated for 1 hr at 37° C. Cells were washed with PBS and RNA was isolated for cfos expression analysis.

Peptide synthesis. The custom peptide library was generated by chemical peptide synthesis at Genscript USA Inc. The library was produced as >70% pure peptides at a quantity of 1-4 mg with a C-terminal amidation as the only modification for comparative studies between peptides. The peptide purity was analyzed by mass spectrometry (theoretical MW). The lyophilized peptides were dissolved in water or DMSO.

RNA expression analysis. Total RNA was isolated using TRIzol (Thermo Fischer Scientific) and Rneasy mini kits (QIAGEN). RNA was reverse transcribed using the ABI high-capacity cDNA synthesis kit. For qRT-PCR analysis, cDNA, primers and SYBR-green fluorescent dye (ABI) was used. Relative mRNA expression was determined by normalization with Ribosomalprotein S18 (Rsp18) levels using the ΔΔCt method. The primer sequences used are CATGCAGAACCCACGACAGTA and CCTCACGCAGCTTGTTGTCTA for Rsp18 and TCTCCTGAAGAGGAAGAGAAACGG and TCTGCAACGCAGACTTCTCG for cfos.

Food intake, energy expenditure and body composition measurements. Measurements of accumulated food intake and VO2 were performed using a Comprehensive Lab Animal Monitoring System at room temperature (20-22° C.) (Oxymax, Columbus Instruments) as previously described. Singly housed mice were acclimated in metabolic cages for at least 24 h before the start of experiment to minimize stress.

Glucose tolerance and insulin tolerance tests. For glucose tolerance tests, mice were fasted for 6 h and I.P. injected with glucose at 1.5 g/kg body weight. Blood glucose levels were measured at 0, 15, 30, 45, 60, 90 and 120 mins. For insulin tolerance tests, mice were fasted for 2 h, and I.P. injected with 0.8 U/kg insulin. Blood glucose levels were measured at 0, 15, 30, 45, 60, 90 and 120 mins.

Immunohistochemistry. For hematoxylin and eosin (H&E) staining, iWAT, BAT and livers were formalin-fixed, paraffin-embedded, and sectioned at 6 μm. Sections were deparaffinized and dehydrated with xylenes and ethanol. Briefly, slides were stained with hematoxylin, washed with water and 95% ethanol, and stained with eosin for 30 min. Sections were then incubated with ethanol and xylene, and mounted with mounting medium. Immunohistochemical stainings were observed with a Nikon 80i upright light microscope using a 40× objective lens. Digital images were captured with a Nikon Digital Sight DS-Fil color camera and NIS-Elements acquisition software.

Open field assay. 8-week-old C57BL/6 mice were transferred to the open-field facility and housed for 7 days for habituation before the test. Mice were weighed and then I.P. injected with either saline or BRP peptide at 5 mg/kg (N=15 mice per group, 10 mg/ml stock solution diluted in saline, total injection volume 100 ul per mouse). 30 min post-injection, the open-field test was performed in an Open Field Activity Arena (Med Associates Inc., St. Albans, VT. Model ENV-515) mounted with three planes of infrared detectors and within a specially designed sound-attenuating chamber (Med Associates Inc., St. Albans, VT. MED-017M-027). The arena was 43 cm (L)×43 cm (W)×30 cm (H), and the sound-attenuating chamber was 74 cm (L)×60 cm (W)×60 cm (H). Each mouse was placed in the corner of the testing arena and allowed to explore the arena for 10 minutes while being tracked by an automated tracking system. Data was analyzed using software Activity Monitor Version 7.8. Parameters analyzed include distance moved and time spent in the periphery and center of the arena. The periphery was defined as the zone 5 cm away from the arena wall.

Statistical analyses. Replicates are described in the figure legends. All values in graphs are presented as mean±SEM. Values for n represent biological replicates for cell experiments or individual animals for in vivo experiments. For animal experiments, n corresponds to the number of animals per condition. Specific details for n values are noted in each figure legend. Mice were randomly assigned to treatment groups for in vivo studies. Each animal experiment was repeated using at least two cohorts of mice. Student's t test was used for single comparisons. Significant differences between two groups (*p<0.05, **p<0.01, ***p<0.001) were evaluated using a two-tailed, unpaired Student's t test as the sample groups displayed a normal distribution and comparable variance. Two-way ANOVA with repeated measures was used for the food intake, body weights, GTT and ITT.
A. Berrington de Gonzalez et al., Body-mass index and mortality among 1.46 million white adults. The New England journal of medicine. 363, 2211-2219 (2010).
J. L. Kuk, C. I. Ardern, T. S. Church, A. M. Sharma, R. Padwal, X. Sui, S. N. Blair, Edmonton Obesity Staging System: association with weight history and mortality risk. Applied physiology, nutrition, and metabolism=Physiologie appliquee, nutrition et metabolisme. 36, 570-576 (2011).
R. R. Wing, W. Lang, T. A. Wadden, M. Safford, W. C. Knowler, A. G. Bertoni, J. O. Hill, F. L. Brancati, A. Peters, L. Wagenknecht, Benefits of modest weight loss in improving cardiovascular risk factors in overweight and obese individuals with type 2 diabetes. Diabetes care. 34, 1481-1486 (2011).
S. O'Rahilly, I. S. Farooqi, Human obesity as a heritable disorder of the central control of energy balance. International journal of obesity (2005). 32 Suppl 7, S55-61 (2008).
D. Polidori, A. Sanghvi, R. J. Seeley, K. D. Hall, How Strongly Does Appetite Counter Weight Loss?Quantification of the Feedback Control of Human Energy Intake. Obesity (Silver Spring, Md.). 24, 2289-2295 (2016).
D. O. Magro, B. Geloneze, R. Delfini, B. C. Pareja, F. Callejas, J. C. Pareja, Long-term weight regain after gastric bypass: a 5-year prospective study. Obesity surgery. 18, 648-651 (2008).
C. H. Lin, L. Shao, Y. M. Zhang, Y. J. Tu, Y. Zhang, B. Tomlinson, P. Chan, Z. Liu, An evaluation of liraglutide including its efficacy and safety for the treatment of obesity. Expert opinion on pharmacotherapy. 21, 275-285 (2020).
J. P. H. Wilding, R. L. Batterham, S. Calanna, M. Davies, L. F. Van Gaal, I. Lingvay, B. M. McGowan, J. Rosenstock, M. T. D. Tran, T. A. Wadden, S. Wharton, K. Yokote, N. Zeuthen, R. F. Kushner, Once-Weekly Semaglutide in Adults with Overweight or Obesity. The New England journal of medicine. 384, 989-1002 (2021).
C. Sobrino Crespo, A. Perianes Cachero, L. Puebla Jimenez, V. Barrios, E. Arilla Ferreiro, Peptides and food intake. Frontiers in endocrinology. 5, 58 (2014).
J. E. Campbell, D. J. Drucker, Pharmacology, physiology, and mechanisms of incretin hormone action. Cell metabolism. 17, 819-837 (2013).
M. Muttenthaler, G. F. King, D. J. Adams, P. F. Alewood, Trends in peptide drug discovery. Nature Reviews Drug Discovery. 20, 309-325 (2021).
H. G. Pollock, J. W. Hamilton, J. B. Rouse, K. E. Ebner, A. B. Rawitch, Isolation of peptide hormones from the pancreas of the bullfrog (Rana catesbeiana). Amino acid sequences of pancreatic polypeptide, oxyntomodulin, and two glucagon-like peptides. The Journal of biological chemistry. 263, 9746-9751 (1988).
I. Vecchio, C. Tornali, N. L. Bragazzi, M. Martini, The discovery of insulin: An important milestone in the history of medicine. Frontiers in Endocrinology. 9 (2018), p. 613.
K. Tatemoto, Neuropeptide Y: complete amino acid sequence of the brain peptide. Proceedings of the National Academy of Sciences of the United States of America. 79, 5485-5489 (1982).
D. A. Lovejoy, W. H. Fischer, S. Ngamvongchon, A. G. Craig, C. S. Nahorniak, R. E. Peter, J. E. Rivier, N. M. Sherwood, Distinct sequence of gonadotropin-releasing hormone (GnRH) in dogfish brain provides insight into GnRH evolution. Proceedings of the National Academy of Sciences of the United States of America. 89, 6373-6377 (1992).
K. L. Lee, M. J. Middleditch, G. M. Williams, M. A. Brimble, G. J. S. Cooper, Using Mass Spectrometry to Detect, Differentiate, and Semiquantitate Closely Related Peptide Hormones in Complex Milieu: Measurement of IGF-II and Vesiculin. Endocrinology. 156, 1194-1199 (2015).
J. E. Lee, Neuropeptidomics: Mass Spectrometry-Based Identification and Quantitation of Neuropeptides. Genomics & informatics. 14, 12-19 (2016).
L. D. Fricker, J. Lim, H. Pan, F.-Y. Che, Peptidomics: Identification and quantification of endogenous peptides in neuroendocrine tissues. Mass Spectrometry Reviews. 25, 327-344 (2006).
B. Muthusamy, et al., Plasma Proteome Database as a resource for proteomics research. Proteomics. 5, 3531-3536 (2005).
J. M. Schwenk, G. S. Omenn, Z. Sun, D. S. Campbell, M. S. Baker, C. M. Overall, R. Aebersold, R. L. Moritz, E. W. Deutsch, The Human Plasma Proteome Draft of 2017: Building on the Human Plasma PeptideAtlas from Mass Spectrometry and Complementary Assays. Journal of proteome research. 16, 4299-4310 (2017).
B. R. Southey, A. Amare, T. A. Zimmerman, S. L. Rodriguez-Zas, J. V Sweedler, NeuroPred: a tool to predict cleavage sites in neuropeptide precursors and provide the masses of the resulting peptides. Nucleic acids research. 34, W267-72 (2006).
B. R. Southey, J. V Sweedler, S. L. Rodriguez-Zas, A python analytical pipeline to identify prohormone precursors and predict prohormone cleavage sites. Frontiers in neuroinformatics. 2, 7 (2008).
D. F. Steiner, The proprotein convertases. Current opinion in chemical biology. 2, 31-39 (1998).
M. Zheng, R. D. Streck, R. E. Scott, N. G. Seidah, J. E. Pintar, The developmental expression in rat of proteases furin, PC1, PC2, and carboxypeptidase E: implications for early maturation of proteolytic processing capacity. The Journal of neuroscience: the official journal of the Society for Neuroscience. 14, 4656-4673 (1994).
J. R. Peinado, H. Li, K. Johanning, I. Lindberg, Cleavage of recombinant proenkephalin and blockade mutants by prohormone convertases 1 and 2: an in vitro specificity study. Journal of neurochemistry. 87, 868-878 (2003).
M. Uhlen, et al., Tissue-based map of the human proteome. Science. 347, 1260419 (2015).
R. Petryszak, et al., Expression Atlas update—An integrated database of gene and protein expression in humans, animals and plants. Nucleic Acids Research. 44, D746-D752 (2016).
B. A. Eipper, D. A. Stoffers, R. E. Mains, The biosynthesis of neuropeptides: peptide alpha-amidation. Annual review of neuroscience. 15, 57-85 (1992).
K.-H. Kim, B. L. Seong, Peptide amidation: Production of peptide hormonesin vivo andin vitro. Biotechnology and Bioprocess Engineering. 6, 244-251 (2001).
H. Mukai, K. Kawai, Y. Suzuki, K. Yamashita, E. Munekata, Stimulation of dog gastropancreatic hormone release by neuromedin B and its analogues. The American journal of physiology. 252, E765-71 (1987).
A. Wettergren, L. Pridal, M. Wejdemann, J. J. Holst, Amidated and non-amidated glucagon-like peptide-1 (GLP-1): non-pancreatic effects (cephalic phase acid secretion) and stability in plasma in humans. Regulatory peptides. 77, 83-87 (1998).
J. Milbrandt, Nerve growth factor rapidly induces c-fos mRNA in PC12 rat pheochromocytoma cells. Proceedings of the National Academy of Sciences of the United States of America. 83, 4789-4793 (1986).
N. Gabellini, M. C. Minozzi, A. Leon, R. Dal Toso, Nerve growth factor transcriptional
control of c-fos promoter transfected in cultured spinal sensory neurons. The Journal of cell biology. 118, 131-138 (1992).
D. J. Drucker, Mechanisms of Action and Therapeutic Application of Glucagon-like Peptide-1. Cell Metabolism. 27, 740-756 (2018).
S. R. Berkowicz, T. J. Featherby, J. C. Whisstock, P. I. Bird, Mice Lacking Brinp2 or Brinp3, or Both, Exhibit Behaviors Consistent with Neurodevelopmental Disorders. Frontiers in behavioral neuroscience. 10, 196 (2016).
J. T. Kim, S. M. Terrell, V. L. Li, W. Wei, C. R. Fischer, J. Z. Long, Cooperative enzymatic control of N-acyl amino acids by PM20D1 and FAAH. eLife. 9 (2020), doi:10.7554/eLife.55211.
J. T. Kim, V. L. Li, S. M. Terrell, C. R. Fischer, J. Z. Long, Family-wide Annotation of Enzymatic Pathways by Parallel In Vivo Metabolomics. Cell chemical biology. 26, 1623-1629.e3 (2019).
P. Cohen, et al., Ablation of PRDM16 and beige adipose causes metabolic dysfunction and a subcutaneous to visceral fat switch. Cell. 156, 304-316 (2014).
J. Z. Long, et al., Ablation of PM20D1 reveals N-acyl amino acid control of metabolism and nociception. Proceedings of the National Academy of Sciences of the United States of America. 115, E6937-E6945 (2018).
K. J. Svensson, J. Z. Long, M. P. Jedrychowski, P. Cohen, J. C. Lo, S. Serag, S. Kir, K. Shinoda, J. A. Tartaglia, R. R. Rao, A. Chédotal, S. Kajimura, S. P. Gygi, B. M. Spiegelman, A secreted slit2 fragment regulates adipose tissue thermogenesis and metabolic function. Cell Metabolism. 23, 454-466 (2016).
Z. Jiang, M. Zhao, L. Voilquin, Y. Jung, M. A. Aikio, T. Sahai, F. Y. Dou, A. M. Roche, I. Carcamo-Orive, J. W. Knowles, M. Wabitsch, E. A. Appel, C. L. Maikawa, J. P. Camporez, G. I. Shulman, L. Tsai, E. D. Rosen, C. D. Gardner, B. M. Spiegelman, K. J. Svensson, Isthmin-1 is an adipokine that promotes glucose uptake and improves glucose tolerance and hepatic steatosis. Cell metabolism (2021), doi:10.1016/j.cmet.2021.07.010.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in protein structure without affecting the biological action in kind or amount. All such modifications are intended to be included within the scope of the appended claims.

1. An isolated peptide comprising or consisting essentially of the amino acid sequence of SEQ ID NO: 2:

| | | | | | | | | | | | |
|---|---|---|---|---|---|---|---|---|---|---|---|
| T₁ | H₂ | R₃ | I₄ | L₅ | R₆ | R₇ | L₈ | X₉ | N₁₀ | L₁₁ | C₁₂ |

where X⁹ is any amino acid, with the proviso that when X⁹ is F, the peptide comprises a non-naturally occurring modification.

2. The peptide of claim 1, comprising at least one amidated amino acid.

3. The peptide of claim 2, wherein the amidated amino acid is located at one or more of C-terminal carboxyl group, C₁₂ thiol, R₃, R₆ and R₇.

4. The peptide of claim 1, comprising at least one acylated residue.

5. The peptide of claim 4, comprising a C₄-C₂₀ alkyl fatty acid.

6. The peptide of claim 5, wherein the fatty acid is palmitate.

7. The peptide of claim 1, wherein the peptide is modified by pegylation, glycosylation, conjugation to albumin, or conjugation to an immunoglobulin Fc.

8. An isolated peptide comprising a sequence according to any of SEQ ID NO:9-20.

9. A method of providing therapeutic weight loss or managing body weight of a subject comprising administering to the subject an isolated peptide comprising or consisting essentially of the amino acid sequence of SEQ ID NO: 2:

| | | | | | | | | | | | |
|---|---|---|---|---|---|---|---|---|---|---|---|
| T₁ | H₂ | R₃ | I₄ | L₅ | R₆ | R₇ | L₈ | X₉ | N₁₀ | L₁₁ | C₁₂ |

where X⁹ is any amino acid.

10. A pharmaceutical formulation comprising an isolated peptide according to claim 1, and a pharmaceutically acceptable excipient.

11. The pharmaceutical formulation of claim 10, in a unit dose.

12. A method of managing body weight in a subject in need thereof the method comprising: administering to the subject an effective dose of a pharmaceutical formulation comprising an isolated peptide according to claim 1.

13. The method of claim 12, wherein the effective dose in the range of from about 0.1 mg/kg to about 100 mg/kg.

14. The method of claim 12, wherein the administering comprises parental dosing.

15. The method of claim 12, wherein the subject is overweight or obese.

16. The method of claim 12, wherein the subject has been diagnosed with metabolic syndrome or type II diabetes.

17. The method of claim 12, wherein the subject reduces food intake relative to an untreated subject.

18. The method of claim 12, wherein the subject is concomitantly treated with a therapeutic agent selected from the group consisting of an antidiabetic agent, an anti-obesity agent, an anti-hypertensive agent, an anti-atherosclerotic agent and a lipid-lowering agent.
